SEQUENCE LISTING (72 sequences)

SEQ ID NO: 1 - protein, 16 aa, Artificial Sequence
  Synthetic
RSSQSLLHSDGYNYLD

SEQ ID NO: 2 - protein, 7 aa, Artificial Sequence
  Synthetic
LGSNRAS

SEQ ID NO: 3 - protein, 9 aa, Artificial Sequence
  Synthetic
MQALQTPWT

SEQ ID NO: 4 - protein, 11 aa, Artificial Sequence
  Synthetic
RASQGISNWLA

SEQ ID NO: 5 - protein, 7 aa, Artificial Sequence
  Synthetic
AASSLQS

SEQ ID NO: 6 - protein, 9 aa, Artificial Sequence
  Synthetic
QQANSFPLT

SEQ ID NO: 7 - protein, 5 aa, Artificial Sequence
  Synthetic
SYDMH

SEQ ID NO: 8 - protein, 17 aa, Artificial Sequence
  Synthetic
VIWYDGSNKYYADSLKG

SEQ ID NO: 9 - protein, 14 aa, Artificial Sequence
  Synthetic
VIAARTFYYYGMDV

SEQ ID NO: 10 - protein, 5 aa, Artificial Sequence
  Synthetic
NYWMS

SEQ ID NO: 11 - protein, 17 aa, Artificial Sequence
  Synthetic
NIKKDGSEKFYVDSVKG

SEQ ID NO: 12 - protein, 8 aa, Artificial Sequence
  Synthetic
EIQLYLQH

SEQ ID NO: 13 - protein, 23 aa, Artificial Sequence
  Synthetic
DIVMTQSPLSLPVTPGEPASISC

SEQ ID NO: 14 - protein, 15 aa, Artificial Sequence
  Synthetic
WYLQKSGQSPQLLIY

SEQ ID NO: 15 - protein, 32 aa, Artificial Sequence
  Synthetic
GVPDRFSGSGSGTDFTLKISRVEAEDVGVYYC

SEQ ID NO: 16 - protein, 11 aa, Artificial Sequence
  Synthetic
FGQGTKVEIKR

SEQ ID NO: 17 - protein, 23 aa, Artificial Sequence
  Synthetic
DIQMTQSPSSVSASVGGRVTITC

SEQ ID NO: 18 - protein, 15 aa, Artificial Sequence
  Synthetic
WYQQKPGKAPKLLIY

SEQ ID NO: 19 - protein, 32 aa, Artificial Sequence
  Synthetic
GVPSRFSGSGSGTDFTLTISNLQPEDFASYYC

SEQ ID NO: 20 - protein, 11 aa, Artificial Sequence
  Synthetic
FGGGTKVEIKR

SEQ ID NO: 21 - protein, 30 aa, Artificial Sequence
  Synthetic
QVQLVESGGGVVQPGRSLRLSCAASGFTFS

SEQ ID NO: 22 - protein, 14 aa, Artificial Sequence
  Synthetic
WVRQAPGKGLEWVA

SEQ ID NO: 23 - protein, 32 aa, Artificial Sequence
  Synthetic
RFTISRDNSKNTLYLQMNSLRAEDTAVYYCAR

SEQ ID NO: 24 - protein, 11 aa, Artificial Sequence
  Synthetic
WGQGTTVTVSS

SEQ ID NO: 25 - protein, 30 aa, Artificial Sequence
  Synthetic
EVQLVESGGGLVQPGGSLRLSCAASGITFS

SEQ ID NO: 26 - protein, 14 aa, Artificial Sequence
  Synthetic
WVRQAPGKGLEWVA

SEQ ID NO: 27 - protein, 32 aa, Artificial Sequence
  Synthetic
RFTISRDNAKNSLYLQINSLRAEDTAMYYCAR

SEQ ID NO: 28 - protein, 11 aa, Artificial Sequence
  Synthetic
WGQGTLVTVSS

SEQ ID NO: 29 - protein, 219 aa, Artificial Sequence
  Synthetic
DIVMTQSPLSLPVTPGEPASISCRSSQSLLHSDGYNYLDWYLQKSGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, Artificial Sequence
  Synthetic
DIQMTQSPSSVSASVGGRVTITCRASQGISNWLAWYQQKPGKAPKLLIYAASSLQSGVPSRFSGSGSGTDFTLTISNLQPEDFASYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 453 aa, Artificial Sequence
  Synthetic
QVQLVESGGGVVQPGRSLRLSCAASGFTFSSYDMHWVRQAPGKGLEWVAVIWYDGSNKYYADSLKGRFTISRDNSKNTLYLQMNSLRAEDTAVYYCARVIAARTFYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 447 aa, Artificial Sequence
  Synthetic
EVQLVESGGGLVQPGGSLRLSCAASGITFSNYWMSWVRQAPGKGLEWVANIKKDGSEKFYVDSVKGRFTISRDNAKNSLYLQINSLRAEDTAMYYCAREIQLYLQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 657 bp, Artificial Sequence
  Synthetic
gatattgtgatgactcagtctccactctccctgcccgtcacccctggagagccggcctccatctcctgcaggtctagtcagagcctcctgcatagtgatggatacaactatttggattggtacctgcagaagtcagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - DNA, 642 bp, Artificial Sequence
  Synthetic
gacatccagatgacccagtctccttcttccgtgtctgcatctgtaggaggcagagtcaccatcacttgtcgggcgagtcagggtattagcaactggttagcctggtatcagcagaaaccagggaaagcccctaaactcctgatctatgctgcatccagtttgcaaagtggggtcccatcaaggttcagcggcagtggatctgggacagatttcactctcaccatcagcaacctgcagcctgaagattttgcaagttactattgtcaacaggctaacagtttccccctcactttcggcggagggaccaaggtggagatcaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 35 - DNA, 1359 bp, Artificial Sequence
  Synthetic
caggtgcagctggtggagtctgggggaggcgtggtccagcctgggaggtccctgagactctcctgtgcagcgtctggattcaccttcagtagctatgacatgcactgggtccgccaggctccaggcaaggggctggagtgggtggcagttatttggtatgatggaagtaataaatactatgcagactccttgaagggccgattcaccatctccagagacaattccaagaacacgctgtatctgcaaatgaacagcctcagagccgaggacacggctgtgtattactgtgcgagggttatagcagctcgtaccttctactactacggtatggacgtctggggccaagggaccacggtcaccgtctcctcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - DNA, 1341 bp, Artificial Sequence
  Synthetic
gaggtgcagctggtggagtctgggggaggcttggtccagcctggggggtccctgagactctcctgtgcagcctctggaatcacctttagtaattattggatgagctgggtccgccaggctccagggaagggactggagtgggtggccaacataaagaaagatggaagtgagaaattctatgtggactctgtgaagggccgattcaccatctccagagacaacgccaagaactcactgtatctgcaaatcaacagcctgagagccgaggacacggctatgtattactgtgcgagagaaatacagctatacctgcagcactggggccagggcaccctggtcaccgtctcctcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - protein, 11 aa, Artificial Sequence
  Synthetic
RASQSISRFLN

SEQ ID NO: 38 - protein, 7 aa, Artificial Sequence
  Synthetic
VTSSLQS

SEQ ID NO: 39 - protein, 9 aa, Artificial Sequence
  Synthetic
QQSYNTPIT

SEQ ID NO: 40 - protein, 11 aa, Artificial Sequence
  Synthetic
RASQSIGTFLN

SEQ ID NO: 41 - protein, 7 aa, Artificial Sequence
  Synthetic
VASSLQS

SEQ ID NO: 42 - protein, 9 aa, Artificial Sequence
  Synthetic
QQSYSVPIT

SEQ ID NO: 43 - protein, 5 aa, Artificial Sequence
  Synthetic
SYVMH

SEQ ID NO: 44 - protein, 17 aa, Artificial Sequence
  Synthetic
GVSSSGDSTFYVDSVKG

SEQ ID NO: 45 - protein, 12 aa, Artificial Sequence
  Synthetic
GGITGAPLVFDI

SEQ ID NO: 46 - protein, 5 aa, Artificial Sequence
  Synthetic
SHIMY

SEQ ID NO: 47 - protein, 17 aa, Artificial Sequence
  Synthetic
GISSNGLSSYYVDSVKG

SEQ ID NO: 48 - protein, 12 aa, Artificial Sequence
  Synthetic
GGRDRVPAVFDY

SEQ ID NO: 49 - protein, 23 aa, Artificial Sequence
  Synthetic
DIQMTQSPSSLSASVGDRVTITC

SEQ ID NO: 50 - protein, 15 aa, Artificial Sequence
  Synthetic
WYQQKPGKAPKVLIY

SEQ ID NO: 51 - protein, 32 aa, Artificial Sequence
  Synthetic
GVPSRFSGSGSGTDFTLTISSLQPEDFATYYC

SEQ ID NO: 52 - protein, 11 aa, Artificial Sequence
  Synthetic
FGQGTRLEIKR

SEQ ID NO: 53 - protein, 23 aa, Artificial Sequence
  Synthetic
DIQMTQSPSSLSASIGDRVTITC

SEQ ID NO: 54 - protein, 15 aa, Artificial Sequence
  Synthetic
WYQQKPGKAPKVLIY

SEQ ID NO: 55 - protein, 32 aa, Artificial Sequence
  Synthetic
GVPSRFIGSGSGTDFTLTISSLQPEDFATYYC

SEQ ID NO: 56 - protein, 11 aa, Artificial Sequence
  Synthetic
FGQGTRLEIKR

SEQ ID NO: 57 - protein, 30 aa, Artificial Sequence
  Synthetic
EVQLVESGGGLVQPGGSLRLSCAASGFTFS

SEQ ID NO: 58 - protein, 14 aa, Artificial Sequence
  Synthetic
WVRQAPGKGLEYVS

SEQ ID NO: 59 - protein, 32 aa, Artificial Sequence
  Synthetic
RFTISRDNSKNTLYLQMGSLRAEDMAVYYCAR

SEQ ID NO: 60 - protein, 11 aa, Artificial Sequence
  Synthetic
WGQGTMVTVSS

SEQ ID NO: 61 - protein, 30 aa, Artificial Sequence
  Synthetic
EVQLVESGGGWVQPGGSLRLSCAASGFTFS

SEQ ID NO: 62 - protein, 14 aa, Artificial Sequence
  Synthetic
WVRQAPGKGLEYVS

SEQ ID NO: 63 - protein, 32 aa, Artificial Sequence
  Synthetic
RFTISRDNSKNLLYVHMGSLKPEDMAMYYCAR

SEQ ID NO: 64 - protein, 11 aa, Artificial Sequence
  Synthetic
WGQGTLVTVSS

SEQ ID NO: 65 - protein, 214 aa, Artificial Sequence
  Synthetic
DIQMTQSPSSLSASVGDRVTITCRASQSISRFLNWYQQKPGKAPKVLIYVTSSLQSGVPSRFSGSGSGTDFTLTISSLQPEDFATYYCQQSYNTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, Artificial Sequence
  Synthetic
DIQMTQSPSSLSASIGDRVTITCRASQSIGTFLNWYQQKPGKAPKVLIYVASSLQSGVPSRFIGSGSGTDFTLTISSLQPEDFATYYCQQSYSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 451 aa, Artificial Sequence
  Synthetic
EVQLVESGGGLVQPGGSLRLSCAASGFTFSSYVMHWVRQAPGKGLEYVSGVSSSGDSTFYVDSVKGRFTISRDNSKNTLYLQMGSLRAEDMAVYYCARGGITGAPLVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 451 aa, Artificial Sequence
  Synthetic
EVQLVESGGGWVQPGGSLRLSCAASGFTFSSHIMYWVRQAPGKGLEYVSGISSNGLSSYYVDSVKGRFTISRDNSKNLLYVHMGSLKPEDMAMYYCARGGRDRVPAVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 642 bp, Artificial Sequence
  Synthetic
gacatccagatgacccagtctccatcctccctgtctgcatctgtaggagacagagtcaccatcacttgccgggcaagtcagagcattagcaggtttttaaattggtatcagcagaaaccagggaaagcccctaaggtcctgatctatgttacatccagtttacaaagtggggtcccatcaaggttcagtggcagtggatctgggacagatttcactctcaccatcagcagtctgcaacctgaagattttgcaacttattactgtcaacagagttacaatacccctatcaccttcggccaagggacacgactggagattaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - DNA, 642 bp, Artificial Sequence
  Synthetic
gacatccagatgacccagtctccatcctccctgtctgcatctataggagacagagtcaccatcacttgccgggcaagtcagagcattggcacctttttaaattggtatcaacaaaaaccagggaaagcccctaaggtcctgatctatgttgcatccagtttgcaaagtggggtcccatcaaggttcattggcagtggatctgggacagatttcactctcaccatcagcagtctgcaacctgaagattttgcaacttactactgtcaacagagttacagtgttccgatcaccttcggccaagggacacgactggagattaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 71 - DNA, 1353 bp, Artificial Sequence
  Synthetic
gaggtgcagctggtggagtctgggggaggcttggtccagcctggggggtccctgagactctcctgtgcagcctctggattcaccttcagtagttatgttatgcactgggtccgccaggctccagggaagggactggaatatgtttcaggtgttagtagtagtggggatagcacattttatgtagactctgtgaagggcagattcaccatctccagagacaattccaagaacacgctttatcttcaaatgggcagcctgagagctgaggacatggctgtgtattactgtgcgagagggggtataactggagctccactggtttttgatatctggggccaagggacaatggtcaccgtctcttcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 72 - DNA, 1353 bp, Artificial Sequence
  Synthetic
gaggtgcaactggtggagtctgggggaggctgggtccagccgggggggtccctgagactctcctgtgcagcctctggattcaccttcagtagtcatattatgtactgggtccgccaggctccagggaagggactggaatatgtttcgggtattagcagtaatggacttagctcatattatgttgactctgtgaagggcagattcaccatctccagagacaattccaagaatttactgtatgttcatatgggcagcctgaaacctgaggacatggctatgtattactgtgcgagagggggccgggatagagtgccagctgtctttgactactggggccagggaaccctggtcaccgtctcctccgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2023/0071763 A1
(43) Pub. Date: Mar. 9, 2023

(54) AURISTATIN-RELATED COMPOUNDS, CONJUGATED AURISTATIN-RELATED COMPOUNDS, AND METHODS OF USE THEREOF

(71) Applicant: CYTOMX THERAPEUTICS, INC., South San Francisco, CA (US)

(72) Inventors: Siew Schleyer, South San Francisco, CA (US); Brian A. Mendelsohn, South San Francisco, CA (US); Pia Challita-Eid, South San Francisco, CA (US); Dowdy Jackson, South San Francisco, CA (US); Christopher Kemball, South San Francisco, CA (US)

(21) Appl. No.: 17/790,835

(22) PCT Filed: Jan. 6, 2021
(86) PCT No.: PCT/US2021/012364

§ 371 Date: Jul. 5, 2022

Related U.S. Application Data
(60) Provisional application No. 62/957,780, filed on Jan. 6, 2020.

(51) Int. Cl. C07K 5/02; A61K 47/68

(52) U.S. Cl. CPC C07K 5/0205; A61K 47/68

(57) ABSTRACT

The invention relates generally to novel compounds of the auristatin family, to novel linkers for coupling a payload to another molecule, such a target-binding molecule, to novel linker-toxin molecules, and to novel antibody molecules that allow controlled, site-specific conjugation.

### CROSS-REFERENCE TO RELATED APPLICATIONS

The invention claims the benefit of U.S. Provisional Application No. 62/957,780, filed on Jan. 6, 2020, the contents of which are incorporated herein by reference in their entirety.

### FIELD OF THE INVENTION

The present disclosure relates generally to novel compounds of the auristatin family. The present disclosure also generally relates to novel linkers for coupling a payload to another molecule, such a target-binding molecule. The present disclosure also generally relates to novel linker-toxin molecules. The present disclosure relates to target-binding molecules conjugated to novel linker-toxin molecules, where the toxin is a novel compound of the auristatin family.

### REFERENCE TO SEQUENCE LISTING

The Sequence Listing submitted electronically concurrently herewith pursuant 37 C.F.R. § 1.821 in computer readable form (ASCII format) via EFS-Web as file name CYTX_070_PCT_ST25.txt is incorporated herein by reference. The ASCII copy of the Sequence Listing was created on Jan. 6, 2021 and is 48 kilobytes in size.

### BACKGROUND OF THE INVENTION

Several short peptidic compounds, known as dolastatins, have been isolated from natural sources or and found to have antimitotic biological activity by binding to and blocking the polymerization of tubulin. Analogs of these compounds, known as auristatins, have also been prepared, and some were found to have similar activity.

Such molecules are used therapeutically by conjugating them via a chemical linker to a target-binding moiety, such as a target-specific monoclonal antibody, thereby delivering the toxic payload in a target-specific manner. The efficacy and safety of such molecules can depend on the nature of the toxin and the stability of the connecting linker, as linkers with low stability will release the drug in situ, thereby potentially increasing the toxicity and tolerability of the drug.

Conjugation of drug to antibodies or activatable antibodies typically rely on chemical reactions that link the drug to amino or thiol side chains on the heavy or light chains. However, reliance on these native amino acid residues may result in varying stoichiometries between the drug and the antibody (DAR) after conjugation, or the need to reduce the antibody to break existing cysteine disulfide bonds to allow conjugation.

Accordingly, there is a continued need in the field of drugs with suitable efficacy and sufficiently stable linkers. There is also a continued need in the field for novel antibody variants that allow controlled, site-specific conjugation.

### BRIEF SUMMARY OF THE INVENTION

Provided herein are compounds of formulae (I), (II), and (III);

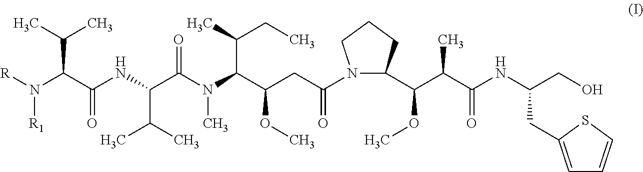

wherein R1 is a hydrogen or a C₁₋₆ alkyl group and wherein R is selected from the group consisting of: a hydrogen, a C₁₋₆ alkyl, a linker, or a group X1-Y1-* wherein * is the point of attachment to the nitrogen,

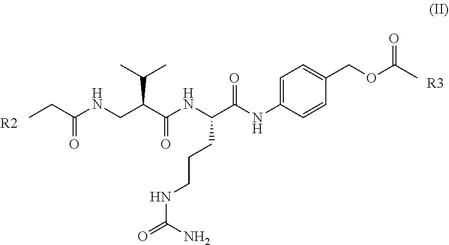

wherein R3 is an agent attached to formula (II) where the point of attachment is a nitrogen, sulfur, oxygen, or carbon atom and wherein R2 is a moiety attached to formula (II) wherein the point of attachment is selected from the group consisting of: a chlorine group, an iodine group, a bromine group, and a thiol group,

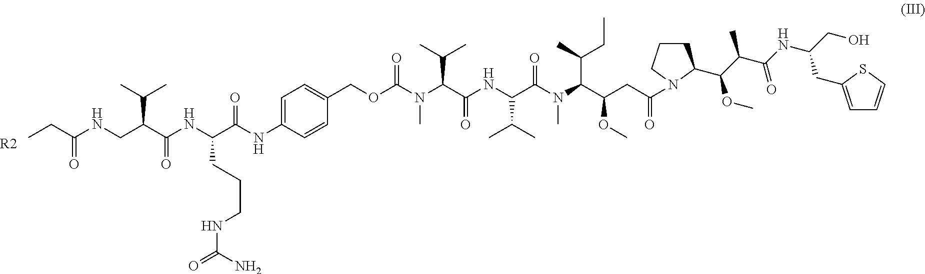

wherein R2 is a moiety attached to formula (III) wherein the point of attachment is selected from the group consisting of: a chlorine group, an iodine group, a bromine group, and a thiol group.

Provided herein are antibodies and activatable antibodies wherein Kabat position 328 is a cysteine. In some embodiments, the compounds of formulae (I), (II), and (III) are conjugated to a polypeptide. In some embodiments, the compounds of formulae (I), (II), or (III) are conjugated to an antibody to a side chain thiol group of a cysteine at Kabat position 328.

In some embodiments of the compound of formula (I) of the present disclosure, Y1 is an oxycarbonyl group and X1 is a C₁₋₆ alkyl group, a 9-fluorenylmethyl group, a benzyl group, or a tert-butyl group. In some embodiments of the compound of formula (I), R1 is a methyl group and R is a hydrogen. In some embodiments of the compound of formula (I), X1-Y1 is a 9-fluorenylmethoxycarbonyl (Fmoc) group.

In some embodiments of the compound of formula (II) of the present disclosure, R2 is a target-binding moiety, wherein the point of attachment at R2 is a thiol group. In some embodiments of the compound of formula (II), the target-binding moiety is an isolated antibody or an antigen binding fragment thereof (AB) that specifically binds to the target. In some embodiments of the compound of formula (II), the target-binding moiety is an activatable antibody that, in an activated state, specifically binds to the target, and the activatable antibody includes an antibody or an antigen binding fragment thereof (AB) that specifically binds to the target, a masking moiety (MM) coupled to the AB, wherein the MM inhibits the binding of the AB to the target when the activatable antibody is in an uncleaved state, a cleavable moiety (CM) coupled to the AB, wherein the CM is a polypeptide that functions as a substrate for a protease. In some embodiments of formula (II), the MM has a dissociation constant for binding to the AB that is greater than the dissociation constant of the AB to its target, the MM does not interfere or compete with the AB for binding to its target when the activatable antibody is in a cleaved state, the MM is a polypeptide of no more than 40 amino acids in length, the MM polypeptide sequence is different from that of the target sequence, and/or the MM polypeptide sequence is no more than 50% identical to any natural binding partner of the AB. In some embodiments of formula (II), the target is selected from the group consisting of CD44, CD147, CD166, ITGa3, ITGb1, PSMA, and SLC34A2. In some embodiments of formula (II), the agent is selected from the group consisting of auristatin E, monomethyl auristatin F (MMAF), monomethyl auristatin E (MMAE), monomethyl auristatin D (MMAD), maytansinoid DM4, maytansinoid DM1, a calicheamicin, a duocarmycin, a pyrrolobenzodiazepine, and a pyrrolobenzodiazepine dimer

In some embodiments of formula (I), R is a linker. In some embodiments, the linker is a cleavable linker. In some embodiments, the linker is linked to a target-binding moiety. In some embodiments, the target-binding moiety is an antibody or antigen binding fragment thereof. In some embodiments, the target is selected from the group consisting of CD44, CD147, CD166, ITGa3, ITGb1, PSMA, and SLC34A2. In some embodiments, the antibody or activatable antibody comprises a cysteine residue at Kabat position 328.

In some embodiments, the compound of formula (I), (II), or (III) is linked to a polypeptide to a thiol group. In some embodiments, the thiol group is a thiol group side chain of a cysteine residue. In some embodiments, the cysteine residue is a cysteine residue at Kabat position 328 of an antibody.

In some embodiments of the present disclosure, a method of conjugating a method of conjugating a compound to a polypeptide, the method comprising conjugating a compound of formula (I) to a polypeptide, wherein R1 is a hydrogen or a C1-6 alkyl group, wherein R is selected from the group consisting of: a hydrogen, a C1-6 alkyl, a linker, or a group X1-Y1-* wherein * is the point of attachment to the nitrogen; and wherein Y1 is an oxycarbonyl group and X1 is a C1-6 alkyl group, a 9-fluorenylmethyl group, a benzyl group, or a tert-butyl group, wherein at least one equivalent of the compound of formula (I) or a derivative thereof is conjugated to the polypeptide.

In some embodiments of the present disclosure, a method of conjugating a method of conjugating a compound to a polypeptide, the method comprising conjugating a compound of formula (II) to a polypeptide, wherein R2 is a moiety attached to formula (II) wherein the point of attachment is selected from the group consisting of: a chlorine group, an iodine group, a bromine group, and a thiol group.

In some embodiments of the present disclosure, a method of conjugating a method of conjugating a compound to a polypeptide comprises reducing the polypeptide with a reducing agent, wherein at least one disulfide group is reduced to a free thiol group, re-oxidizing the polypeptide with an oxidizing agent without oxidizing the free thiol group, and conjugating the compound of formula (I) or (III) to the free thiol group.

### BRIEF DESCRIPTION OF THE DRAWINGS

FIG. 1 is a schematic overview of a synthetic path to auristatin species of the present disclosure.

FIGS. 2A and 2B show graphs depicting the exemplary in vitro stability of a linker of the present disclosure to activated cathepsin B.

FIGS. 3A and 3B show graphs depicting exemplary in vitro stability of a linker of the present disclosure to activated lysosomes.

FIG. 4 shows a process flow diagram of an exemplary method of linker-toxin activation and conjugation of the linker-toxin to an antibody.

### DETAILED DESCRIPTION OF THE INVENTION

The present disclosure relates generally to novel compounds of the auristatin family. The present disclosure also generally relates to novel linkers for coupling a payload to another molecule, such a target-binding molecule. The present disclosure also generally relates to novel linker-toxin molecules. Examples of such embodiments are described in the examples below.

In some embodiments, a target-binding moiety to which compounds of the present disclosure can be conjugated include anti-PSMA antibodies, examples of which are described in the sequences below:

Table 1
VL CDR Amino Acid Sequences

| | VL CDR1 | VL CDR2 | VL CDR3 |
|---|---|---|---|
| | (SEQ ID | (SEQ ID | (SEQ ID |
| Antibody | NO:) | NO:) | NO:) |
| [Ag]AB-4 | SEQUENCE | SEQUENCE | SEQUENCE |
| cHv75-2a11.G1 | (SEQ ID | (SEQ ID | (SEQ ID |
| (L328C)k | NO: 1) | NO: 2) | NO: 3) |
| | RSSQSLLH | LGSNRAS | MQALQT |
| | SDGYNYLD | | PWT |
| [Ag]AB-5 | SEQUENCE | SEQUENCE | SEQUENCE |
| cHv75-2a7.G1 | (SEQ ID | (SEQ ID | (SEQ ID |
| (C99Y; | NO: 4) | NO: 5) | NO: 6) |
| L328C)k | RASQGIS | AASSIQS | QQANSF |
| | NWIA | | PLT |

TABLE 2
VH CDR Amino Add Sequences

| | VH CDR1 | VH CDR2 | VH CDR3 |
|---|---|---|---|
| | (SEQ ID | (SEQ ID | (SEQ ID |
| Antibody | NO:) | NO:) | NO:) |
| [Ag]AB-4 | SE- | SE- | SE- |
| cHv75- | QUENCE | QUENCE | QUENCE |
| 2a11.Gl | (SEQ ID | (SEQ ID | (SEQ ID |
| (L328C)k | NO: 7) | NO: 8) | NO: 9) |
| | SYDMH | VIWYDG | VIAART |
| | | SNKYYA | FYYYGM |
| | | DSLKG | DV |
| [Ag]AB-5 | SE- | SE- | SE- |
| cH75- | QUENCE | QUENCE | QUENCE |
| 2a7.G1 | (SEQ ID | (SEQ ID | (SEQ ID |
| (C99Y;k | NO: 10) | NO: 11) | NO: 12) |
| L328C) | NYWMS | NIKKDGSE | EIQLYLOH |
| | | KFYVDSVKG | |

TABLE 3
VL FR Amino Add Sequences

| | VL FR1 | VL FR2 | VL FR3 | VL FR4 |
|---|---|---|---|---|
| | (SEQ ID | (SEQ ID | (SEQ ID | (SEQ ID |
| Antibody | NO:) | NO:) | NO:) | NO:) |
| [Ag]AB-4 | SE- | SE- | SE- | SE- |
| CHV75- | QUENCE | QUENCE | QUENCE | QUENCE |
| 2a11.G1 | (SEQ ID | (SEQ ID | (SEQ ID | (SEQ ID |
| (1328C)k | NO: 13) | NO: 14) | NO: 15) | NO: 16) |
| | DIVMTQSP | WYLQK | GVPDRFSG | FGQGTKV |
| | LSLPVTPG | SGQSP | SGSGTDFT | EIKR |
| | EPASISC | QLLIY | LKISRVEA | |
| | | | EDVGVYYC | |
| | SE- | SE- | SE- | SE- |
| [Ag] | QUENCE | QUENCE | QUENCE | QUENCE |
| AB-5 | (SEQ ID | (SEQ ID | (SEQ ID | (SEQ ID |
| cHv75- | NO: 17) | NO: 18) | NO: 19) | NO: 20) |
| 2a7.G1 | DIQMTQSP | WYQQKP | GVPSRFS | FGGGTKV |
| (C99Y; | SSVSASV | GKAPKL | GSGSGTD | EIKR |
| L328C)k | GGRVTITC | LIY | FTLTISN | |
| | | | LQPEDFA | |
| | | | SYYC | |

TABLE 4
VH FR Amino Acid Sequences

| | VH FR1 | VH FR2 | VH FR3 | VH FR4 |
|---|---|---|---|---|
| | (SEQ ID | (SEQ ID | (SEQ ID | (SEQ ID |
| Antibody | NO:) | NO:) | NO:) | NO:) |
| [Ag] | SE- | SE- | SE- | SE- |
| AB-4 | QUENCE | QUENCE | QUENCE | QUENCE |
| cHv75- | (SEQ ID | (SEQ ID | (SEQ ID | (SEQ ID |
| 2a11.G1 | NO: 21) | NO: 22) | NO: 23) | NO: 24) |
| (L32 | QVQLVES | WVRQAPGK | RFTISRD | WGQGTT |
| 8C)k | GGGVVQP | GLEWVA | NSKNTLY | VTVSS |
| | GRSLRLS | | LQMNSLR | |
| | CAASGFT | | AEDTAVY | |
| | FS | | YCAR | |
| [Ag] | SE- | SE- | SE- | SE- |
| AB-5 | QUENCE | QUENCE | QUENCE | QUENCE |
| cHv75- | (SEQ ID | (SEQ ID | (SEQ ID | (SEQ ID |
| 2a7.G1 | NO: 25) | NO: 26) | NO: 27) | NO: 28) |
| (C99Y; | EVQLVES | WVRQAP | RFTISRD | WGQGTL |
| 1328C)k | GGGLVQP | GKGLE | NAKNSLYL | VTVSS |
| | GGSLRLS | WVA | QINSLRAE | |
| | CAASGIT | | DTAMYYC | |
| | FS | | AR | |

TABLE 5
VL Domain Amino Add Sequences
Variable region (double underline), constant region (dotted underline)

| Antibody | VL (SEQ ID NO:) |
|---|---|
| [Ag] AB-4 cHv75- 2a11.G1 (L328C)k | SEQUENCE (SEQ ID NO: 29) 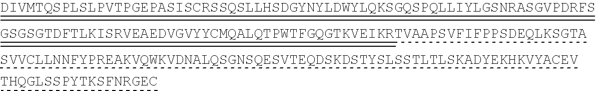 |
| [Ag] AB-5 cHv75- 2a7.G1 (C99Y; L328C)k | SEQUENCE (SEQ ID NO: 30) 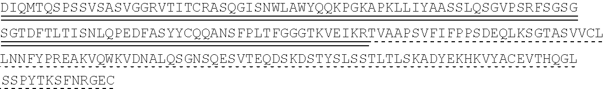 |

TABLE 6
VH Domain Amino Acid Sequences
Variable region (double underline), constant region (dotted underline)

| Antibody | VH (SEQ ID NO:) |
|---|---|
| [Ag] AB-4 cHv75- 2a11.G1 (L328C)k | SEQUENCE (SEQ ID NO: 31) 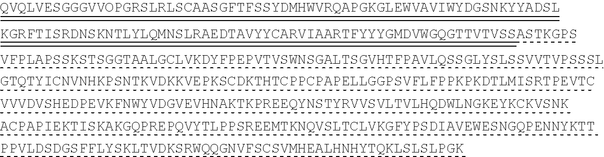 |
| [Ag] AB-5 cHv75- 2a7.G1 (C99Y; L328C)k | SEQUENCE (SEQ ID NO: 32) 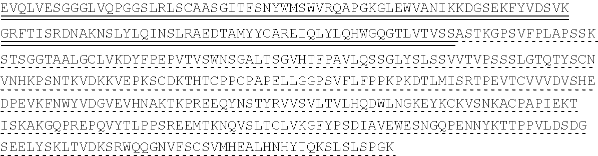 |

TABLE 7
VL Nucleic Acid Sequences

| Antibody | Nucleotide sequences |
|---|---|
| [Ag]A | SEQUENCE |
| B-4 | (SEQ ID NO: 33) |
| cHv7 | GATATTGTGATGACTCAGTCTCCAC |
| 5- | TCTCCCTGCCCGTCACCCCTGGAGA |
| 2a11. | GCCGGCCTCCATCTCCTGCAGGTCT |
| G1(L | AGTCAGAGCCTCCTGCATAGTGATG |
| 328C) | GATACAACTATTTGGATTGGTACCT |
| k | GCAGAAGTCAGGGCAGTCTCCACAG |
| | CTCCTGATCTATTTGGGTTCTAATC |
| | GGGCCTCCGGGGTCCCTGACAGGTT |
| | CAGTGGCAGTGGATCAGGCACAGAT |
| | TTTACACTGAAAATCAGCAGAGTGG |
| | AGGCTGAGGATGTTGGGGTTTATTA |
| | CTGCATGCAAGCTCTACAAACTCCG |
| | TGGACGTTCGGCCAAGGGACCAAGG |
| | TGGAAATCAAACGGACTGTCGCTGC |
| | ACCATCTGTCTTCATCTTCCCGCCA |
| | TCTGATGAGCAGTTGAAATCTGGAA |
| | CTGCCTCTGTTGTGTGCCTGCTGAA |
| | TAACTTCTATCCCAGAGAGGCCAAA |
| | GTACAGTGGAAGGTGGATAACGCCC |
| | TCCAATCGGGTAACTCCCAGGAGAG |
| | TGTCACAGAGCAGGACAGCAAGGAC |
| | AGCACCTACAGCCTCAGCAGCACCC |
| | TGACGCTGAGCAAAGCAGACTACGA |
| | GAAACACAAAGTCTACGCCTGCGAA |
| | GTCACCCATCAGGGCCTGAGCTCGC |
| | CCGTCACAAAGAGCTTCAACAGGGG |
| | AGAGTGT |
| [Ag]A | SEQUENCE |
| B-5 | (SEQ ID NO: 34) |
| cHv7 | GACATCCAGATGACCCAGTCTCCTT |
| 5- | CTTCCGTGTCTGCATCTGTAGGAGG |
| 2a7.G | CAGAGTCACCATCACTTGTCGGGCG |
| 1(C99 | AGTCAGGGTATTAGCAACTGGTTAG |
| Y; L3 | CCTGGTATCAGCAGAAACCAGGGAA |
| 28) | AGCCCCTAAACTCCTGATCTATGCT |
| | GCATCCAGTTTGCAAAGTGGGGTCC |
| | CATCAAGGTTCAGCGGCAGTGGATC |
| | TGGGACAGATTTCACTCTCACCATC |
| | AGCAACCTGCAGCCTGAAGATTTTG |
| | CAAGTTACTATTGTCAACAGGCTAA |
| | CAGTTTCCCCCTCACTTTCGGCGGA |
| | GGGACCAAGGTGGAGATCAAACGGA |
| | CTGTCGCTGCACCATCTGTCTTCAT |
| | CTTCCCGCCATCTGATGAGCAGTTG |
| | AAATCTGGAACTGCCTCTGTTGTGT |
| | GCCTGCTGAATAACTTCTATCCCAG |
| | AGAGGCCAAAGTACAGTGGAAGGTG |
| | GATAACGCCCTCCAATCGGGTAACT |
| | CCCAGGAGAGTGTCACAGAGCAGGA |
| | CAGCAAGGACAGCACCTACAGCCTC |
| | AGCAGCACCCTGACGCTGAGCAAAG |
| | CAGACTACGAGAAACACAAAGTCTA |
| | CGCCTGCGAAGTCACCCATCAGGGC |
| | CTGAGCTCGCCCGTCACAAAGAGCT |
| | TCAACAGGGGAGAGTGT |

TABLE 8
VH Nucleic Acid Sequences

| Antibody | Nucleotide sequences |
|---|---|
| [Ag]A | SEQUENCE |
| B-4 | (SEQ ID NO: 35) |
| cHv7 | CAGGTGCAGCTGGTGGAGTCTGGGG |
| 5- | GAGGCGTGGTCCAGCCTGGGAGGTC |
| 2a11. | CCTGAGACTCTCCTGTGCAGCGTCT |
| Gl(L | GGATTCACCTTCAGTAGCTATGACA |
| 328C) | TGCACTGGGTCCGCCAGGCTCCAGG |
| k | CAAGGGGCTGGAGTGGGTGGCAGTT |
| | ATTTGGTATGATGGAAGTAATAAAT |
| | ACTATGCAGACTCCTTGAAGGGCCG |
| | ATTCACCATCTCCAGAGACAATTCC |
| | AAGAACACGCTGTATCTGCAAATGA |
| | ACAGCCTCAGAGCCGAGGACACGGC |
| | TGTGTATTACTGTGCGAGGGTTATA |
| | GCAGCTCGTACCTTCTACTACTACG |
| | GTATGGACGTCTGGGGCCAAGGGAC |
| | CACGGTCACCGTCTCCTCAGCATCC |
| | ACCAAGGGCCCATCGGTCTTCCCCC |
| | TGGCACCCTCCTCCAAGAGCACCTC |
| | TGGGGGCACAGCGGCCCTGGGCTGC |
| | CTGGTCAAGGACTACTTCCCCGAAC |
| | CGGTGACGGTGTCGTGGAACTCAGG |
| | CGCCCTGACCAGCGGCGTGCACACC |
| | TTCCCGGCTGTCCTACAGTCCTCAG |
| | GACTCTACTCCCTCAGCAGCGTGGT |
| | GACCGTGCCCTCCAGCAGCTTGGGC |
| | ACCCAGACCTACATCTGCAACGTGA |
| | ATCACAAGCCCAGCAACACCAAGGT |
| | GGACAAGAAAGTTGAGCCCAAATCT |
| | TGTGACAAAACTCACACATGCCCAC |
| | CGTGCCCAGCACCTGAACTCCTGGG |
| | GGGACCGTCAGTCTTCCTCTTCCCC |
| | CCAAAACCCAAGGACACCCTCATGA |
| | TCTCCCGGACCCCTGAGGTCACATG |
| | CGTGGTGGTGGACGTGAGCCACGAA |
| | GACCCTGAGGTCAAGTTCAACTGGT |
| | ACGTGGACGGCGTGGAGGTGCATAA |
| | TGCCAAGACAAAGCCGCGGGAGGAG |
| | CAGTACAACAGCACGTACCGTGTGG |
| | TCAGCGTCCTCACCGTCCTGCACCA |
| | GGACTGGCTGAATGGCAAGGAGTAC |
| | AAGTGCAAGGTCTCCAACAAAGCCT |
| | GCCCAGCCCCCATCGAGAAAACCAT |
| | CTCCAAAGCCAAAGGGCAGCCCCGA |
| | GAACCACAGGTGTACACCCTGCCCC |
| | CATCCCGGGAGGAGATGACCAAGAA |
| | CCAGGTCAGCCTGACCTGCCTGGTC |
| | AAAGGCTTCTATCCCAGCGACATCG |
| | CCGTGGAGTGGGAGAGCAATGGGCA |
| | GCCGGAGAACAACTACAAGACCACG |
| | CCTCCCGTGCTGGACTCCGACGGCT |
| | CCTTCTTCCTCTATAGCAAGCTCAC |
| | CGTGGACAAGAGCAGGTGGCAGCAG |
| | GGGAACGTCTTCTCATGCTCCGTGA |
| | TGCATGAGGCTCTGCACAACCACTA |
| | CACGCAGAAGAGCCTCTCCCTGTCT |
| | CCGGGTAAA |
| [Ag]A | SEQUENCE |
| B-5 | (SEQ ID NO: 36) |
| cHv7 | GAGGTGCAGCTGGTGGAGTCTGGGG |
| 2a7.G | GAGGCTTGGTCCAGCCTGGGGGGTC |
| (C99 | CCTGAGACTCTCCTGTGCAGCCTCT |
| Y;L3 | GGAATCACCTTTAGTAATTATTGGA |
| 28C) | TGAGCTGGGTCCGCCAGGCTCCAGG |
| | GAAGGGACTGGAGTGGGTGGCCAAC |
| | ATAAAGAAAGATGGAAGTGAGAAAT |
| | TCTATGTGGACTCTGTGAAGGGCCG |
| | ATTCACCATCTCCAGAGACAACGCC |
| | AAGAACTCACTGTATCTGCAAATCA |
| | ACAGCCTGAGAGCCGAGGACACGGC |
| | TATGTATTACTGTGCGAGAGAAATA |
| | CAGCTATACCTGCAGCACTGGGGCC |
| | AGGGCACCCTGGTCACCGTCTCCTC |
| | AGCATCCACCAAGGGCCCATCGGTC |
| | TTCCCCCTGGCACCCTCCTCCAAGA |
| | GCACCTCTGGGGGCACAGCGGCCCT |
| | GGGCTGCCTGGTCAAGGACTACTTC |
| | CCCGAACCGGTGACGGTGTCGTGGA |
| | ACTCAGGCGCCCTGACCAGCGGCGT |
| | GCACACCTTCCCGGCTGTCCTACAG |
| | TCCTCAGGACTCTACTCCCTCAGCA |
| | GCGTGGTGACCGTGCCCTCCAGCAG |
| | CTTGGGCACCCAGACCTACATCTGC |
| | AACGTGAATCACAAGCCCAGCAACA |
| | CCAAGGTGGACAAGAAAGTTGAGCC |
| | CAAATCTTGTGACAAAACTCACACA |
| | TGCCCACCGTGCCCAGCACCTGAAC |
| | TCCTGGGGGGACCGTCAGTCTTCCT |
| | CTTCCCCCCAAAACCCAAGGACACC |
| | CTCATGATCTCCCGGACCCCTGAGG |
| | TCACATGCGTGGTGGTGGACGTGAG |
| | CCACGAAGACCCTGAGGTCAAGTTC |
| | AACTGGTACGTGGACGGCGTGGAGG |
| | TGCATAATGCCAAGACAAAGCCGCG |
| | GGAGGAGCAGTACAACAGCACGTAC |
| | CGTGTGGTCAGCGTCCTCACCGTCC |
| | TGCACCAGGACTGGCTGAATGGCAA |
| | GGAGTACAAGTGCAAGGTCTCCAAC |
| | AAAGCCTGCCCAGCCCCCATCGAGA |
| | AAACCATCTCCAAAGCCAAAGGGCA |
| | GCCCCGAGAACCACAGGTGTACACC |
| | CTGCCCCCATCCCGGGAGGAGATGA |
| | CCAAGAACCAGGTCAGCCTGACCTG |
| | CCTGGTCAAAGGCTTCTATCCCAGC |
| | GACATCGCCGTGGAGTGGGAGAGCA |
| | ATGGGCAGCCGGAGAACAACTACAA |
| | GACCACGCCTCCCGTGCTGGACTCC |
| | GACGGCTCCTTCTTCCTCTATAGCA |
| | AGCTCACCGTGGACAAGAGCAGGTG |
| | GCAGCAGGGGAACGTTTCTCATGCT |
| | CCGTGATGCATGAGGCTCTGCACAA |
| | CCACTACACGCAGAAGAGCCTCTCC |
| | CTGTCTCCGGGTAAA |

In some embodiments, a target-binding moiety to which compounds of the present disclosure can be conjugated include anti-SLC34A2 antibodies, examples of which are described in the sequences below:

TABLE 9
VL CDR Amino Acid Sequences

| | VL CDR1 | VL CDR2 | VL CDR3 |
|---|---|---|---|
| | (SEQ | (SEQ | (SEQ |
| Antibody | ID NO:) | ID NO:) | ID NO:) |
| [Ag]AB-2 | SEQUENCE | SEQUENCE | SEQUENCE |
| cHv83-3a23. | (SEQ ID | (SEQ ID | (SEQ ID |
| G1(L328C)k | NO: 37) | NO: 38) | NO: 39) |
| | RASQSIS | VTSSLQS | QQSYNT |
| | RFLN | | PIT |
| [Ag]AB-3 | SEQUENCE | SEQUENCE | SEQUENCE |
| cHV83-1b15. | (SEQ ID | (SEQ ID | (SEQ ID |
| G1(L328C)k | NO: 40) | NO: 41) | NO: 42) |
| | RASQSI | VASS | QQSY |
| | GTFLN | LQS | SVPIT |

TABLE 10
VH CDR Amino Acid Sequences

| | VH CDR1 | VH CDR2 | VH CDR3 |
|---|---|---|---|
| | (SEQ | (SEQ | (SEQ |
| Antibody | ID NO:) | ID NO:) | ID NO:) |
| [Ag]AB-2 | SEQUENCE | SEQUENCE | SEQUENCE |
| cHv83- | (SEQ ID | (SEQ ID | (SEQ ID |
| 3a23.G1 | NO: 43) | NO: 44) | NO: 45) |
| (328C)k | SYVMH | GVSSSG | GGITGA |
| | | DSTFYV | PLVFDI |
| | | DSVKG | |
| [Ag]AB-3 | SEQUENCE | SEQUENCE | SEQUENCE |
| cHv83- | (SEQ ID | (SEQ ID | (SEQ ID |
| 1b15.G1 | NO: 46) | NO: 47) | NO: 48) |
| (L328C)k | SHIMY | GISSNGLS | GGRDRV |
| | | SYYVDSVKG | PAVFDY |

TABLE 11
VL FR Amino Acid Sequences

| | VL FR1 | VL FR2 | VL FR3 | VL FR4 |
|---|---|---|---|---|
| | (SEQ ID | (SEQ ID | (SEQ ID | (SEQ ID |
| Antibody | NO:) | NO:) | NO:) | NO:) |
| [Ag]AB-2 | SEQUENCE | SE- | SEQUENCE | SE- |
| cHv83- | (SEQ ID | QUENCE | (SEQ ID | QUENCE |
| 3a23.G1 | NO: 49) | (SEQ ID | NO: 51) | (SEQ |
| (L32 | DIQMTQS | NO: 50) | GVPSRFSG | ID NO: |
| 8C)k | PSSLSAS | WYQQKPG | SGSGTDFT | 52) |
| | VGDRVTI | KAPKVLIY | LTISSLQ | FGQGTRL |
| | TC | | PEDFATYYC | EIKR |
| [Ag]AB-3 | SEQUENCE | SEQUENCE | SEQUENCE | SEQUEN |
| cllv83- | (SEQ ID | (SEQ ID | (SEQ ID | CE |
| 1b15.G1 | NO: 53) | NO: 5 4) | NO: 55) | (SEQ |
| (L3 | DIQMTQS | WYQQKPGKA | GVPSRFIG | ID NO: |
| 28C)k | PSSLSAS | PKVLIY | SGSGTDRL | 56) |
| | IGDRCTI | | TISSLQPE | FGQGTRL |
| | TC | | DFATYYC | EIKR |

TABLE 12
VH FR Amino Acid Sequences

| | VH FR1 | VH FR2 | VH FR3 | VH FR4 |
|---|---|---|---|---|
| | (SEQ ID | (SEQ ID | (SEQ ID NO:) | (SEQ ID |
| Antibody | NO:) | NO:) | | NO:) |
| [Ag]AB-2 | SEQUENCE | SE- | SE- | SE- |
| cHv83- | (SEQ ID | QUENCE | QUENCE | QUENCE |
| 3a23.G | NO: 57) | (SEQ ID | (SEQ ID | (SEQ ID |
| 1(L3 | EVQLV | NO: 58) | NO: 59) | NO: 60) |
| 28C)k | ESGGGL | WVRQ | RFTISR | WGQGTM |
| | VQPGGS | APGK | DNSKNT | VTVSS |
| | LRLSCA | GLEY | LYLQMG | |
| | ASGFTF | VS | SLRAED | |
| | S | | MAVYYC | |
| | | | AR | |
| Antibody | VH FR1 | VH FR2 | VH FR3 | VH FR4 |
| | (SEQ ID | (SEQ ID | (SEQ ID | (SEQ ID |
| | NO:) | NO:) | NO:) | NO:) |
| [Ag]AB-3 | SEQUENCE | SEQUENCE | SEQUENCE | SE- |
| cHv83- | (SEQ ID | (SEQ ID | (SEQ ID | QUENCE |
| lbl5.G1 | NO: 61) | NO: 62) | NO: 63) | (SEQ ID |
| (L328C) | EVQLVE | WVRQ | RFTISR | NO: 64) |
| | SGGGW | APGK | DNSKNL | WGQGTL |
| | VQPGG | GLEY | LYVHMG | VTVSS |
| | SLRLS | VS | SLKPED | |
| | CAASG | | MAMYYC | |
| | FTFS | | AR | |

TABLE 13
VL Domain Amino Acid Sequences
Variable region (double underline), constant region (dotted underline)

| Antibody | VL (SEQ ID NO:) |
|---|---|
| [Ag] AB-2 cHv83- 3a23.G1 (L328C)k | SEQUENCE (SEQ ID NO: 65) 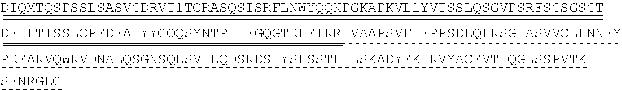 |
| [Ag] AB-3 cHv83- 1b15.G1 (L328C)k | SEQUENCE (SEQ ID NO: 66) 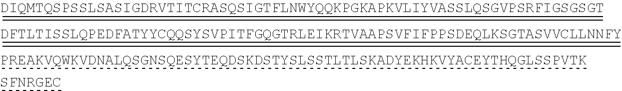 |

TABLE 14
VH Domain Amino Acid Sequences
Variable region (double underline), constant region (dotted underline)

| Antibody | VH (SEQ ID NO:) |
|---|---|
| [Ag] AB-2 cHv83- 3a23.G1 (L328C)k | SEQUENCE (SEQ ID NO: 67) 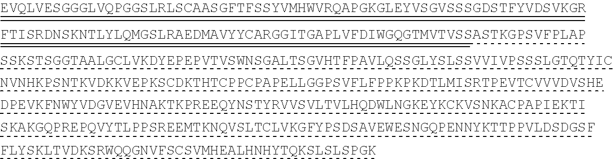 |
| [Ag] AB-3 cHv83- 1b15.G1 (L328C)k | SEQUENCE (SEQ ID NO: 68) 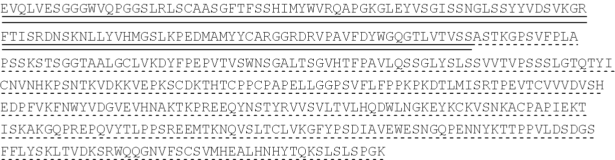 |

TABLE 15
VL Nucleic Acid Sequences

| Antibody | Nucleotide sequences |
|---|---|
| [Ag] | SEQUENCE |
| AB- | (SEQ ID NO: 69) |
| 2 | GACATCCAGATGACCCAGTCTCCAT |
| cHv8 | CCTCCCTGTCTGCATCTGTAGGAGA |
| 3- | CAGAGTCACCATCACTTGCCGGGCA |
| 3a23 | AGTCAGAGCATTAGCAGGTTTTTAA |
| .G1 | ATTGGTATCAGCAGAAACCAGGGAA |
| (1328 | AGCCCCTAAGGTCCTGATCTATGTT |
| C)k | ACATCCAGTTTACAAAGTGGGGTCC |
| | CATCAAGGTTCAGTGGCAGTGGATC |
| | TGGGACAGATTTCACTCTCACCATC |
| | AGCAGTCTGCAACCTGAAGATTTTG |
| | CAACTTATTACTGTCAACAGAGTTA |
| | CAATACCCCTATCACCTTCGGCCAA |
| | GGGACACGACTGGAGATTAAACGGA |
| | CTGTCGCTGCACCATCTGTCTTCAT |
| | CTTCCCGCCATCTGATGAGCAGTTG |
| | AAATCTGGAACTGCCTCTGTTGTGT |
| | GCCTGCTGAATAACTTCTATCCCAG |
| | AGAGGCCAAAGTACAGTGGAAGGTG |
| | GATAACGCCCTCCAATCGGGTAACT |
| | CCCAGGAGAGTGTCACAGAGCAGGA |
| | CAGCAAGGACAGCACCTACAGCCTC |
| | AGCAGCACCCTGACGCTGAGCAAAG |
| | CAGACTACGAGAAACACAAAGTCTA |
| | CGCCTGCGAAGTCACCCATCAGGGC |
| | CTGAGCTCGCCCGTCACAAAGAGCT |
| | TCAACAGGGGAGAGTGT |
| [Ag] | SEQUENCE |
| AB- | (SEQ ID NO: 70) |
| 3 | GACATCCAGATGACCCAGTCTCCAT |
| cHv | CCTCCCTGTCTGCATCTATAGGAGA |
| 83- | CAGAGTCACCATCACTTGCCGGGCA |
| 1b15 | AGTCAGAGCATTGGCACCTTTTTAA |
| .G1 | ATTGGTATCAACAAAAACCAGGGAA |
| (L32 | AGCCCCTAAGGTCCTGATCTATGTT |
| SC) | GCATCCAGTTTGCAAAGTGGGGTCC |
| k | CATCAAGGTTCATTGGCAGTGGATC |
| | TGGGACAGATTTCACTCTCACCATC |
| | AGCAGTCTGCAACCTGAAGATTTTG |
| | CAACTTACTACTGTCAACAGAGTTA |
| | CAGTGTTCCGATCACCTTCGGCCAA |
| | GGGACACGACTGGAGATTAAACGGA |
| | CTGTCGCTGCACCATCTGTCTTCAT |
| | CTTCCCGCCATCTGATGAGCAGTTG |
| | AAATCTGGAACTGCCTCTGTTGTGT |
| | GCCTGCTGAATAACTTCTATCCCAG |
| | AGAGGCCAAAGTACAGTGGAAGGTG |
| | GATAACGCCCTCCAATCGGGTAACT |
| | CCCAGGAGAGTGTCACAGAGCAGGA |
| | CAGCAAGGACAGCACCTACAGCCTC |
| | AGCAGCACCCTGACGCTGAGCAAAG |
| | CAGACTACGAGAAACACAAAGTCTA |
| | CGCCTGCGAAGTCACCCATCAGGGC |
| | CTGAGCTCGCCCGTCACAAAGAGCT |
| | TCAACAGGGGAGAGTGT |
| | AGAGGCCAAAGTACAGTGGAAGGTG |
| | GATAACGCCCTCCAATCGGGTAACT |
| | CCCAGGAGAGTGTCACAGAGCAGGA |
| | CAGCAAGGACAGCACCTACAGCCTC |
| | AGCAGCACCCTGACGCTGAGCAAAG |
| | CAGACTACGAGAAACACAAAGTCTA |
| | CGCCTGCGAAGTCACCCATCAGGGC |
| | CTGAGCTCGCCCGTCACAAAGAGCT |
| | TCAACAGGGGAGAGTGT |

TABLE 16
VH Nucleic Acid Sequences

| Antibody | Nucleotide sequences |
|---|---|
| [Ag] | SEQUENCE |
| AB- | (SEQ ID NO: 71) |
| 2 | GAGGTGCAGCTGGTGGAGTC |
| cHv8 | TGGGGGAGGCTTGGTCCAGC |
| 3- | CTGGGGGGTCCCTGAGACTC |
| 3a23. | TCCTGTGCAGCCTCTGGATT |
| G1 | CACCTTCAGTAGTTATGTTA |
| (1328 | TGCACTGGGTCCGCCAGGCT |
| C)k | CCAGGGAAGGGACTGGAATA |
| | TGTTTCAGGTGTTAGTAGTA |
| | GTGGGGATAGCACATTTTAT |
| | GTAGACTCTGTGAAGGGCAG |
| | ATTCACCATCTCCAGAGACA |
| | ATTCCAAGAACACGCTTTAT |
| | CTTCAAATGGGCAGCCTGAG |
| | AGCTGAGGACATGGCTGTGT |
| | ATTACTGTGCGAGAGGGGGT |
| | ATAACTGGAGCTCCACTGGT |
| | TTTTGATATCTGGGGCCAAG |
| | GGACAATGGTCACCGTCTCT |
| | TCAGCATCCACCAAGGGCCC |
| | ATCGGTCTTCCCCCTGGCAC |
| | CCTCCTCCAAGAGCACCTCT |
| | GGGGGCACAGCGGCCCTGGG |
| | CTGCCTGGTCAAGGACTACT |
| | TCCCCGAACCGGTGACGGTG |
| | TCGTGGAACTCAGGCGCCCT |
| | GACCAGCGGCGTGCACACCT |
| | TCCCGGCTGTCCTACAGTCC |
| | TCAGGACTCTACTCCCTCAG |
| | CAGCGTGGTGACCGTGCCCT |
| | CCAGCAGCTTGGGCACCCAG |
| | ACCTACATCTGCAACGTGAA |
| | TCACAAGCCCAGCAACACCA |
| | AGGTGGACAAGAAAGTTGAG |
| | CCCAAATCTTGTGACAAAAC |
| | TCACACATGCCCACCGTGCC |
| | CAGCACCTGAACTCCTGGGG |
| | GGACCGTCAGTCTTCCTCTT |
| | CCCCCCAAAACCCAAGGACA |
| | CCCTCATGATCTCCCGGACC |
| | CCTGAGGTCACATGCGTGGT |
| | GGTGGACGTGAGCCACGAAG |
| | ACCCTGAGGTCAAGTTCAAC |
| | TGGTACGTGGACGGCGTGGA |
| | GGTGCATAATGCCAAGACAA |
| | AGCCGCGGGAGGAGCAGTAC |
| | AACAGCACGTACCGTGTGGT |
| | CAGCGTCCTCACCGTCCTGC |
| | ACCAGGACTGGCTGAATGGC |
| | AAGGAGTACAAGTGCAAGGT |
| | CTCCAACAAAGCCTGCCCAG |
| | CCCCCATCGAGAAAACCATC |
| | TCCAAAGCCAAAGGGCAGCC |
| | CCGAGAACCACAGGTGTACA |
| | CCCTGCCCCCATCCCGGGAG |
| | GAGATGACCAAGAACCAGGT |
| | CAGCCTGACCTGCCTGGTCA |
| | AAGGCTTCTATCCCAGCGAC |
| | ATCGCCGTGGAGTGGGAGAG |
| | CAATGGGCAGCCGGAGAACA |
| | ACTACAAGACCACGCCTCCC |
| | GTGCTGGACTCCGACGGCTC |
| | CTTCTTCCTCTATAGCAAGC |
| | TCACCGTGGACAAGAGCAGG |
| | TGGCAGCAGGGGAACGTCTT |
| | CTCATGCTCCGTGATGCATG |
| | AGGCTCTGCACAACCACTAC |
| | ACGCAGAAGAGCCTCTCCCT |
| | GTCTCCGGGTAAA |
| [Ag] | SEQUENCE |
| AB- | (SEQ ID NO: 72) |
| 3 | GAGGTGCAACTGGTGGAGTC |
| cHv | TGGGGGAGGCTGGGTCCAGC |
| 83- | CGGGGGGGTCCCTGAGACTC |
| 1b15. | TCCTGTGCAGCCTCTGGATT |
| G1 | CACCTTCAGTAGTCATATTA |
| (L32 | TGTACTGGGTCCGCCAGGCT |
| 8C) | CCAGGGAAGGGACTGGAATA |
| k | TGTTTCGGGTATTAGCAGTA |
| | ATGGACTTAGCTCATATTAT |
| | GTTGACTCTGTGAAGGGCAG |
| | ATTCACCATCTCCAGAGACA |
| | ATTCCAAGAATTTACTGTAT |
| | GTTCATATGGGCAGCCTGAA |
| | ACCTGAGGACATGGCTATGT |
| | ATTACTGTGCGAGAGGGGGC |
| | CGGGATAGAGTGCCAGCTGT |
| | CTTTGACTACTGGGGCCAGG |
| | GAACCCTGGTCACCGTCTCC |
| | TCCGCTTCCACCAAGGGCCC |
| | ATCGGTCTTCCCCCTGGCAC |
| | CCTCCTCCAAGAGCACCTCT |
| | GGGGGCACAGCGGCCCTGGG |
| | CTGCCTGGTCAAGGACTACT |
| | TCCCCGAACCGGTGACGGTG |
| | TCGTGGAACTCAGGCGCCCT |
| | GACCAGCGGCGTGCACACCT |
| | TCCCGGCTGTCCTACAGTCC |
| | TCAGGACTCTACTCCCTCAG |
| | CAGCGTGGTGACCGTGCCCT |
| | CCAGCAGCTTGGGCACCCAG |
| | ACCTACATCTGCAACGTGAA |
| | TCACAAGCCCAGCAACACCA |
| | AGGTGGACAAGAAAGTTGAG |
| | CCCAAATCTTGTGACAAAAC |
| | TCACACATGCCCACCGTGCC |
| | CAGCACCTGAACTCCTGGGG |
| | GGACCGTCAGTCTTCCTCTT |
| | CCCCCCAAAACCCAAGGACA |
| | CCCTCATGATCTCCCGGACC |
| | CCTGAGGTCACATGCGTGGT |
| | GGTGGACGTGAGCCACGAAG |
| | ACCCTGAGGTCAAGTTCAAC |
| | TGGTACGTGGACGGCGTGGA |
| | GGTGCATAATGCCAAGACAA |
| | AGCCGCGGGAGGAGCAGTAC |
| | AACAGCACGTACCGTGTGGT |
| | CAGCGTCCTCACCGTCCTGC |
| | ACCAGGACTGGCTGAATGGC |
| | AAGGAGTACAAGTGCAAGGT |
| | CTCCAACAAAGCCTGCCCAG |
| | CCCCCATCGAGAAAACCATC |
| | TCCAAAGCCAAAGGGCAGCC |
| | CCGAGAACCACAGGTGTACA |
| | CCCTGCCCCCATCCCGGGAG |
| | GAGATGACCAAGAACCAGGT |
| | CAGCCTGACCTGCCTGGTCA |
| | AAGGCTTCTATCCCAGCGAC |
| | ATCGCCGTGGAGTGGGAGAG |
| | CAATGGGCAGCCGGAGAACA |
| | ACTACAAGACCACGCCTCCC |
| | GTGCTGGACTCCGACGGCTC |
| | CTTCTTCCTCTATAGCAAGC |
| | TCACCGTGGACAAGAGCAGG |
| | TGGCAGCAGGGGAACGTCTT |
| | CTCATGCTCCGTGATGCATG |
| | AGGCTCTGCACAACCACTAC |
| | ACGCAGAAGAGCCTCTCCCT |
| | GTCTCCGGGTAAA |

### Example 1: Exemplary Preparation of Auristatin Species

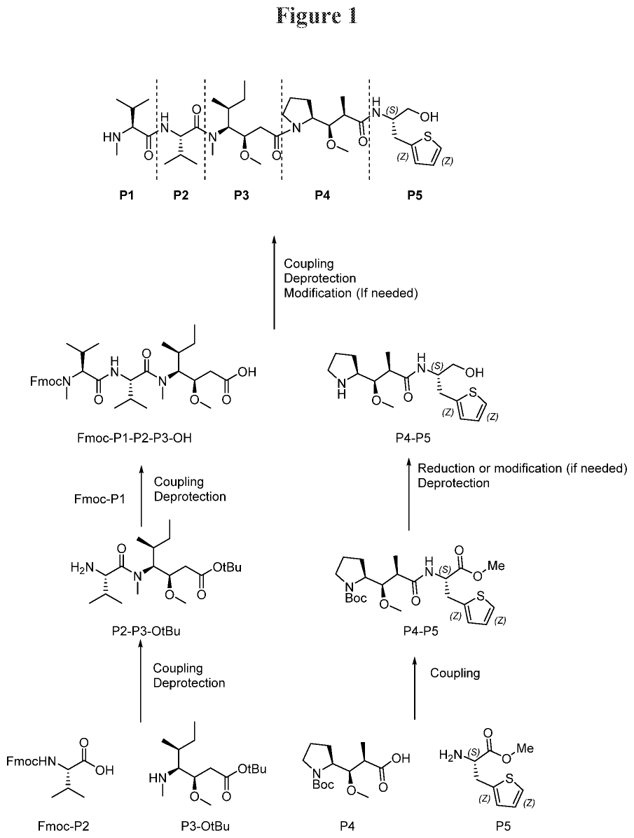

This example provides an exemplary method of preparation of the compound of MMATH (molecule 14), a monomethylauristatin molecule with thiophenylmethyl and hydroxymethyl substituents. A schematic overview of the synthetic preparation of this molecule is depicted in FIG. 1.

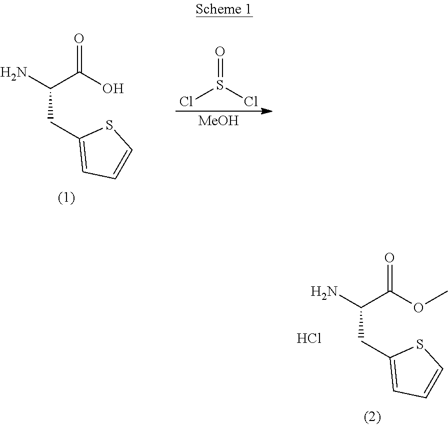

Referring to the reaction outlined in Scheme 1, to a stirred (0° C.) suspension of Ala(2-TH)-OH (molecule 1; 50.04 g, 0.29 mol) in MeOH (500.00 mL) was added SOCl₂ (100.07 mL, 1.38 mol) over 2 hours. The mixture was stirred at 23° C. After 17 h, volatile things were evaporated under reduced pressure. The residue was dried further for 144 hours. Ala(2-Th)-OMe_HCl was obtained (molecule 2). HPLC rt=0.59 min (standard method), ESI [M+H]+ 186.2.

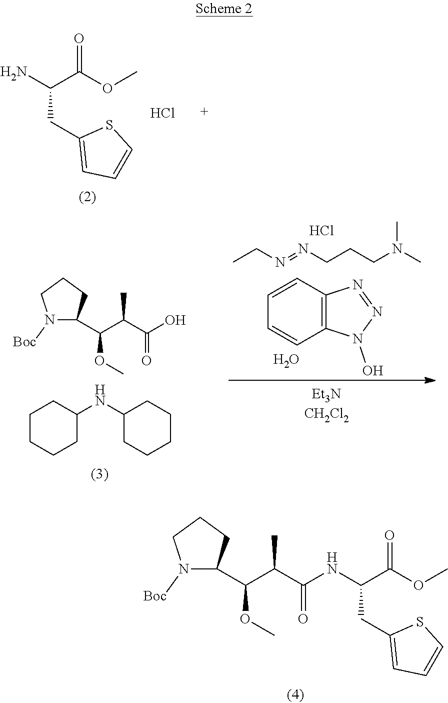

Referring to the reaction outlined in Scheme 2, to a stirred (23° C.) suspension of Ala(2-Th)-OMe_HCl (molecule 2: 64.43 g, 0.29 mol), Boc-Dap-OH_DCHA (molecule 3: 163.64 g, 0.35 mol), WSC_HCl (67.25 g, 0.35 mol) and HOBt_H2O (42.77 g, 0.28 mol) in DCM (1.00 L) was added Et₃N (49.00 mL, 0.35 mol). After 18 h, the reaction mixture was filtered through silica gel pad (approximately 500 g) and filter cake was washed with DCM (1 L). The filtrate was concentrated under reduced pressure until remain was about 500 mL. Undissolved materials were filtered and filter cake was washed with DCM (100 mL). To the filtrate was added 1.0 M HCl aq. (500 mL) and then the mixture was stirred for 30 minutes. After undissolved materials were filtered, the filtrate was separated. The separated organic layer was added 1.0 M HCl aq. (500 mL) again and then the mixture was stirred for 30 minutes. After separation, the organic layer was washed with sat. NaHCO3 aq. (500 mL), Brine (500 mL) and dried over MgSO4. After the organic layer was filtered, the filtrate was concentrated under reduced pressure. The residue was dried further for 3 hours. To the crude material was added AcOEt (200 mL) and then the mixture was heated to 80° C. (internal temperature). The mixture was filtered through Cellite before the filtrate was concentrated under reduce pressure. To the residue was added AcOEt (150 mL) and then the mixture was heat to 80° C. (internal temperature) until materials were dissolved. The mixture was left stand at ambient temperature. After 24 hours, the mixture was filtered and the solid was washed with 50 mL of a 10:1 mixture of Hexane/AcOEt two times. The solid was dried further for 14 hours. Boc-Dap-Ala(2-Th)-OMe (molecule 4; 92.30 g, 0.20 mol) was obtained. HPLC rt=1.52 min (standard method), ESI [M+H]+ 455.2.

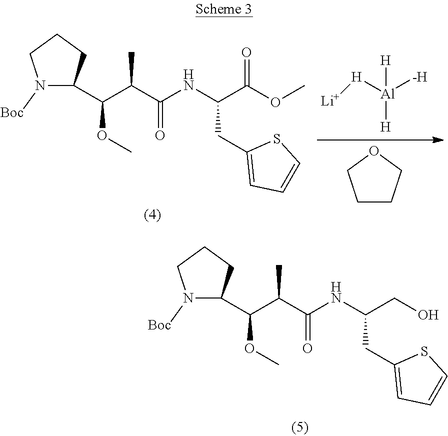

Referring to Scheme 3; under ice-bath cooling, to a stirred solution of LAH (8.25 g, 0.22 mol) in THF (500.00 mL) was added Boc-Dap-Ala(2-Th)-OMe (molecule 4; 39.10 g, 0.09 mol) in THF (100 mL) with maintaining the inner temperature below 5° C. over 2 hours. The reaction mixture was stirred at the same temperature (inner temp; 5° C.). After 5 min, under ice-bath cooling, to the mixture were added H₂O (8.5 mL) slowly, 15% NaOH aq (8.5 mL) and H₂O (25.5 mL) in this order. The mixture was stirred at ambient temperature for 16 hours. The mixture was filtered through a Celite pad and then filter cake was washed with 100 mL of AcOEt three times. The filtrate was concentrated under reduced pressure. The residue was dried further for 4 hours. To the crude material was added Toluene (110 mL) and then the mixture was heated to 60° C. until all materials were dissolved. The mixture was left stand at ambient temperature. After 24 hours, the mixture was filtered and then the solid was washed with 50 mL of Toluene two times and dried further for 15 hours. Boc-Dap-Ala(2-Th)-CH₂OH (molecule 5; 28.43 g, 0.07 mol) was obtained. HPLC rt=1.38 min (standard method), ESI [M+H]+ 427.3.

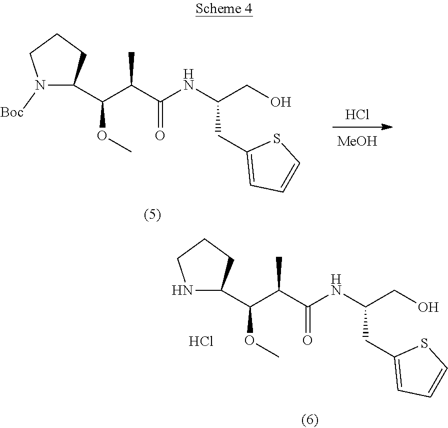 [SMILES: "MeOH" ; CO[C@H]([C@@H](C)C(=O)N[C@H](CO)Cc1cccs1)[C@@H]1CCCN1C(=O)OC(C)(C)C ; Cl ; CO[C@@H]([C@@H]1CCCN1)[C@@H](C)C(=O)N[C@H](CO)Cc1cccs1 ; Cl]

Referring to the reaction outlined in Scheme 4, to a stirred (23° C.) solution of Boc-Dap-Ala(2-Th)-CH₂OH (molecule 5; 19.42 g, 0.05 mol) in MeOH (100.00 mL) was added HCl/dioxane (91.00 mL, 0.36 mol). After 2 h, volatile things were evaporated under reduced pressure. To the residue was added AcOEt (250 mL) and then the mixture was concentrated in vacuo. This process was repeated twice. The residue was dried further for 20 hours. To the crude material was added 20:1 mixture of ACN/H₂O (38 mL). The mixture was heated to 70° C. (internal temperature) until all materials were dissolved, then the mixture was left stand at ambient temperature. After 24 hours, the mixture was filtered and then the solid was washed with 15 mL of ACN two times. The solid was dried further for 8 hours. H-Dap-Ala(2-Th)-CH₂OH_HCl (12.84 g, 0.04 mol) was obtained. HPLC rt=0.60 min (standard method), ESI [M+H]+ 327.2 to a stirred (0° C.) suspension of Ala(2-TH)-OH (50.04 g, 0.29 mol) in MeOH (500.00 mL) was added SOCl₂ (100.07 mL, 1.38 mol) over 2 hours. The mixture was stirred at 23° C. After 17 h, volatile things were evaporated under reduced pressure. The residue was dried further for 144 hours. Ala(2-Th)-OMe_HCl was obtained (molecule 6). HPLC rt=0.59 min (standard method), ESI [M+H]+ 327.2.

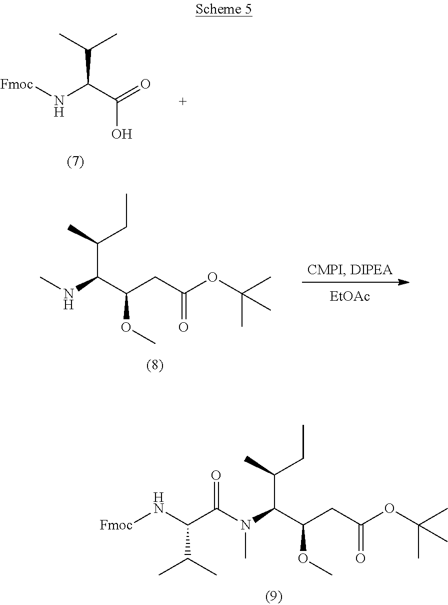

Referring to the reaction outlined in Scheme 5, to a stirred (20° C.) solution of (2S)-2-{([(9H-fluoren-9-ylmethoxy)carbonyl]amino}-3-methylbutanoic acid (molecule 7; 100.00 g, 294.65 mmol), tert-butyl (3R,4S,5S)-3-methoxy-5-methyl-4-(methylamino)heptanoate (molecule 8; 63.69 g, 245.54 mmol), and 2-chloro-1-methylpyridin-1-ium iodide (106.64 g, 417.42 mmol) in ethyl acetate (2.50 L) ethyl acetate (2.50 L) was added N,N-diisopropylethylamine (154.38 mL, 883.95 mmol) once consistent mixing was achieved. After 16 h, the crude reaction mixture was filtered and washed with EtOAc. The solution was extracted with 1 L of 1 M HCl, followed by 1 L of water, followed by 0.5 L sodium bicarbonate, followed by 0.5 L brine. The combined organic fraction was dried using magnesium sulfate, filtered and concentrated under reduced pressure. tert-butyl (3R,4S,5S)-4-[(2S)-2-{[(9H-fluoren-9-ylmethoxy)carbonyl]amino}-N,3-dimethylbutanamido]-3-methoxy-5-methylheptanoate (molecule 9; 149.00 g, 0.26 mol) was obtained as a pink solid. HPLC rt=1.55 min (standard method), ESI [M+H]+ 581.4.

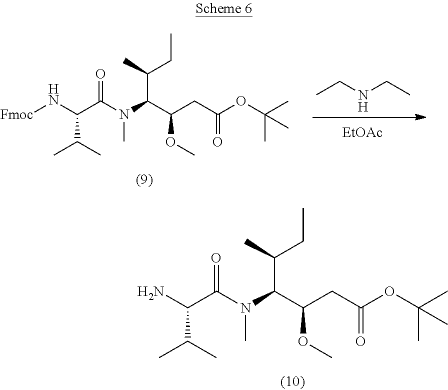

Referring to the reaction outlined in Scheme 6, to a stirred (20° C.) solution of tert-butyl (3R,4S,5S)-4-[(2S)-2-([(9H-fluoren-9-ylmethoxy)carbonyl]amino)-N,3-dimethylbutanamido]-3-methoxy-5-methylheptanoate (molecule 9; 143.00 g, 246.23 mmol) in ethyl acetate (200.00 mL) was added diethylamine (200.00 mL, 1,930.63 mmol). After 1 h, the crude mixture was concentrated in vacuo. The residue was dissolved in 200 mL of ethyl acetate then concentrated again. This operation was repeated twice. To the residue was added 50 mL of toluene, then concentrated. The residue was dissolved in 1000 mL of hexane. To the mixture was added 500 mL of 1 M hydrochloric acid and 500 mL of water. The mixture was stirred for 5 min. The biphasic mixture was put into separation funnel and aqueous layer was separated. The organic layer was extracted by 500 mL of 0.1 M hydrochloric acid twice. The combined aqueous layer was washed with 500 mL of hexane twice. To the aqueous layer was added potassium carbonate to adjust pH over 10. The aqueous solution was put into separation funnel and was extracted by 500 mL of ethyl acetate 3 times. The combined organic layer was washed with 500 mL brine, dried over magnesium sulfate and concentrated in vacuo. tert-butyl (3R,4S,5S)-4-[(2S)-2-amino-N,3-dimethylbutanamido]-3-methoxy-5-methylheptanoate (molecule 10; 66.80 g, 0.19 mol) was obtained as a pink oil. HPLC rt=0.82 min (standard method), ESI [M+H]+ 359.4.

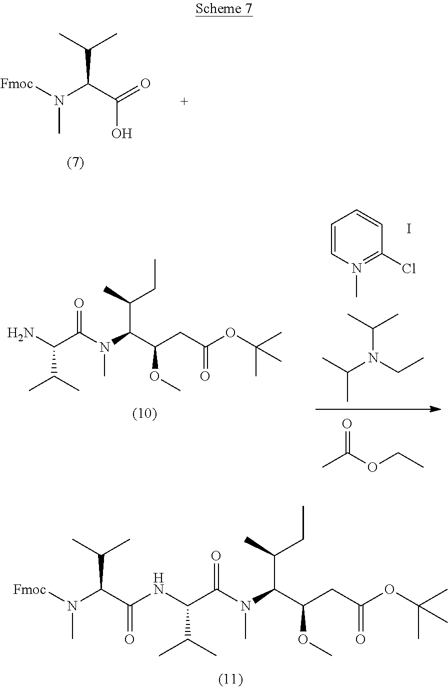

Referring to the reaction outlined in Scheme 7, to a stirred (20° C.) solution of (2S)-2-{[(9H-fluoren-9-ylmethoxy)carbonyl](methyl)amino}-3-methylbutanoic acid (molecule 7; 55.00 g, 155.63 mmol), tert-butyl (3R,4S,5S)-4-[(2S)-2-amino-N,3-dimethylbutanamido]-3-methoxy-5-methylheptanoate (molecule 10; 55.79 g, 0.16 mol), and 2-chloro-1-methylpyridin-1-ium iodide (67.59 g, 264.56 mmol) in ethyl acetate (1.50 L) ethyl acetate (1.50 L) was added N,N-diisopropylethylamine (97.85 mL, 560.25 mmol) once consistent mixing was achieved. After 16 h, the yellow precipitate was removed by celite filtration and washed with 100 mL of EtOAc. The filtrate was put into separation funnel and was washed with 200 mL of 1 M hydrochloric acid twice, 200 mL of water, 200 mL of saturated sodium bicarbonate solution twice and brine. The organic layer was dried over magnesium sulfate and concentrated in vacuo. The residue was dried under hi-vac for 24 hours to give Fmoc-MeVal-Val-Dil-OtBu (molecule 11; 103.53 g, 0.15 mol) as a yellow foam. HPLC rt=1.86 min (standard method), ESI [M+H]+ 694.5.

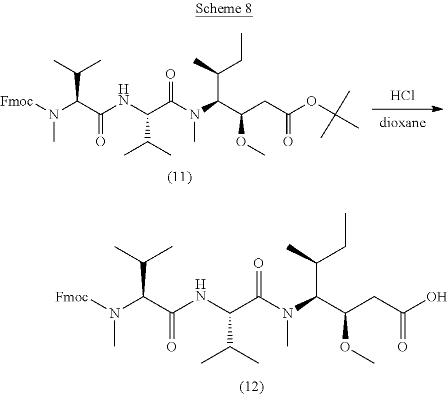

Referring to the reaction outlined in Scheme 8, to a stirred (20° C.) solution of hydrochloric acid (57.64 mL, 230.58 mmol) was added tert-butyl (3R,4S,5S)-4-[(2S)-2-[(2S)-2-{([(9H-fluoren-9-ylmethoxy)carbonyl](methyl)amino}-3-methylbutanamido]-N,3-dimethylbutanamido]-3-methoxy-5-methylheptanoate (molecule 11; 20.00 g, 28.82 mmol). After 16 h, the crude mixture was concentrated in vacuo. The residue was suspended in 50 mL of toluene and concentrated in vacuo. This operation was repeated 3 times. The obtained residue was dried under hi-vac for 24 hours to give Fmoc-MeVal-Val-Dil-OH (molecule 12; 18.00 g, 0.03 mol) as a beige foam. HPLC rt=1.58 min (standard method), ESI [M+H]+ 638.6.

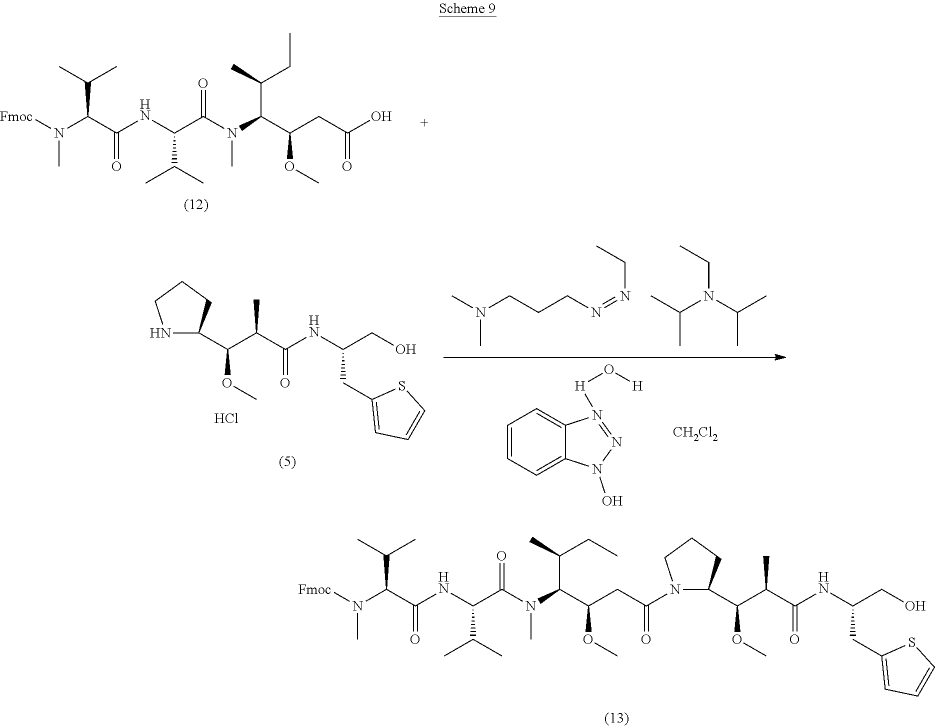

Referring to the reaction outlined in Scheme 9, to Dap-(2-Th)Ala-CH₂OH_HCl (molecule 5; 3.94 g, 10.86 mmol) were added Fmoc-MeVal-Val-Dil-OH (molecule 12; 6.30 g, 9.88 mmol), EDC_HCl (2.84 g, 14.82 mmol), HOBt (1.51 g, 9.88 mmol) and DIPEA (4.30 mL, 24.69 mmol). The reaction mixture was stirred at 23° C. After stirring for 18 h, to the mixture was added CH₂Cl₂ (100 mL). The mixture was washed with 0.1M HCl aq (100 mL), sat. NaHCO₃ aq. (100 mL), then brine (100 mL). The organic layer was dried with MgSO₄ and solid was removed by filtration. The organic layer was concentrated in vacuo to give Fmoc-MMATH (“monomethylauristatin thiophenylmethyl hydroxymethyl) (molecule 13; 7.63 g, 0.01 mol). HPLC rt=1.63 min (standard method), ESI [M+H]+ 946.8.

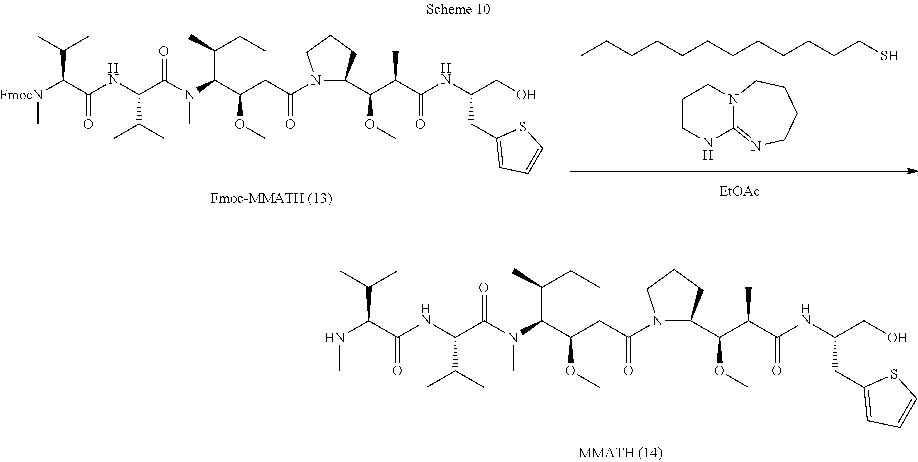

Referring to the reaction outlined in Scheme 10, to Fmoc-MMATH (molecule 13; 7.13 g, 7.54 mmol) were added EtOAc (100.00 mL), dodecyl mercaptan (3.61 mL, 15.07 mmol) and DBU (0.23 mL, 1.51 mmol). The reaction mixture was stirred at 23° C. After stirring for 18 h, the crude mixture was put into separation funnel and was extracted with 50 mL of 1.0 M hydrochloric acid twice. The combined aqueous layer was washed with 100 mL of ethyl acetate twice. The aqueous solution was moved to a round bottom flask. To the mixture was added potassium carbonate to adjust the pH of the mixture over 10. The aqueous solution was put into separation funnel and was extracted with 100 mL of ethyl acetate twice. The combined organic layer was washed with brine, dried over magnesium sulfate and concentrated in vacuo. The residue was dried under hi-vac for 16 hours to give MMATH (molecule 14; 4.51 g, 0.01 mol) as a colorless foam. HPLC rt=0.95 min (standard method), ESI [M+H]+ 724.7.

### Example 2: Exemplary Preparation of Auristatin Linker-Toxin Species

This example provides an exemplary method of preparation of the compound of MMATH (molecule 14), a thiophenylmethyl hydroxymethyl auristatin molecule, with a linker suitable for coupling to a targeting molecule.

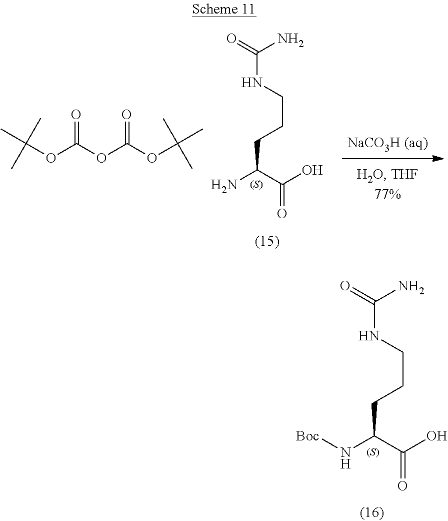

Referring to the reaction outlined in Scheme 11, to a stirred 23° C. solution of Boc₂O (137.0 g, 628 mmol) in THF (600 mL) was added H₂N-Cit-OH (molecule 15; 100.0 g, 571 mmol) and NaCO₃H (71.9 g, 856 mmol) in water (600 mL). After 16 h, a precipitate formed and after 20 h the reaction was complete by LCMS analysis. The volatile organics were removed under reduced pressure and the reaction adjusted to pH 4 2 M HCl and extracted with EtOAC (4×750 mL). The combined organic was washed with Brine and dried with MgSO₄. The solution was filtered and concentrated under reduced pressure to yield 77% of molecule 16 as a white solid.

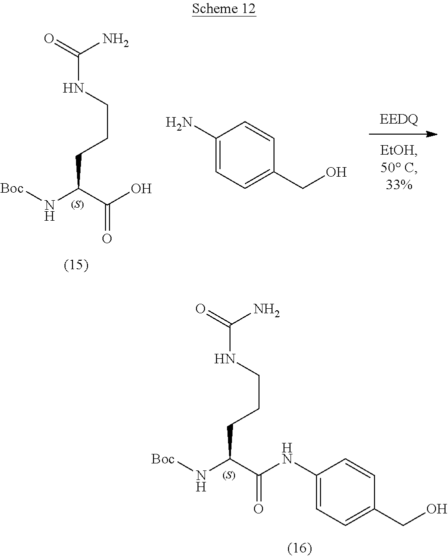

Referring to the reaction outlined in Scheme 12, to a stirred 50° C. solution of Boc-Cit (molecule 16, 120.0 g, 436 mmol) in EtOH (600 mL) was added Paba (64.4 g, 523 mmol) and EEDQ (129.3 g, 523 mmol). The solution was stirred for 24 h and the organic solvents were concentrated to 300 mL. The concentrated crude solution is triturated by adding to 1.0 L of EtOAc followed by addition 2.0 L of Hexanes and stirred for 1 h. The white solid was collected by filtration and dried under reduced pressure to obtained molecule 16 in 77% yield.

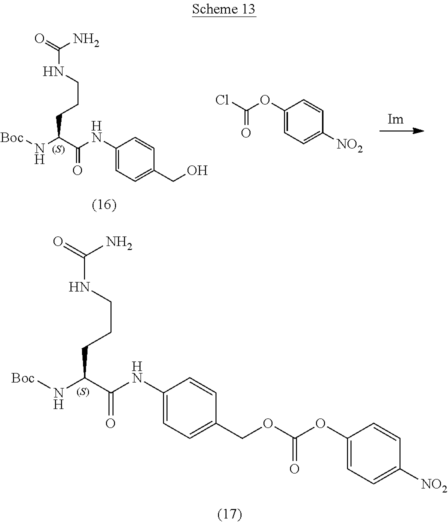

Referring to the reaction outlined in Scheme 13, to a stirred 23° C. solution of Boc-Cit-Paba (molecule 16; 10.0 g, 26.3 mmol) in MeCN (300 mL) was added Im (1.79 g, 26.3 mmol) and then PNP-COCl (7.95 g, 39.4 mmol). After 16 h, the reaction was concentrated under reduced pressure to give a yellow oil. To the oil was added 300 mL of EtOAc and the solution was triturated for 15 minutes. The white precipitate was collected by filtration and the supernatant was concentrated to 50% volume and the second batch triturated for 15 min and collected by filtration. The combined materials were dried under reduced pressure to yield molecule 17 as a white powder in 69% yield.

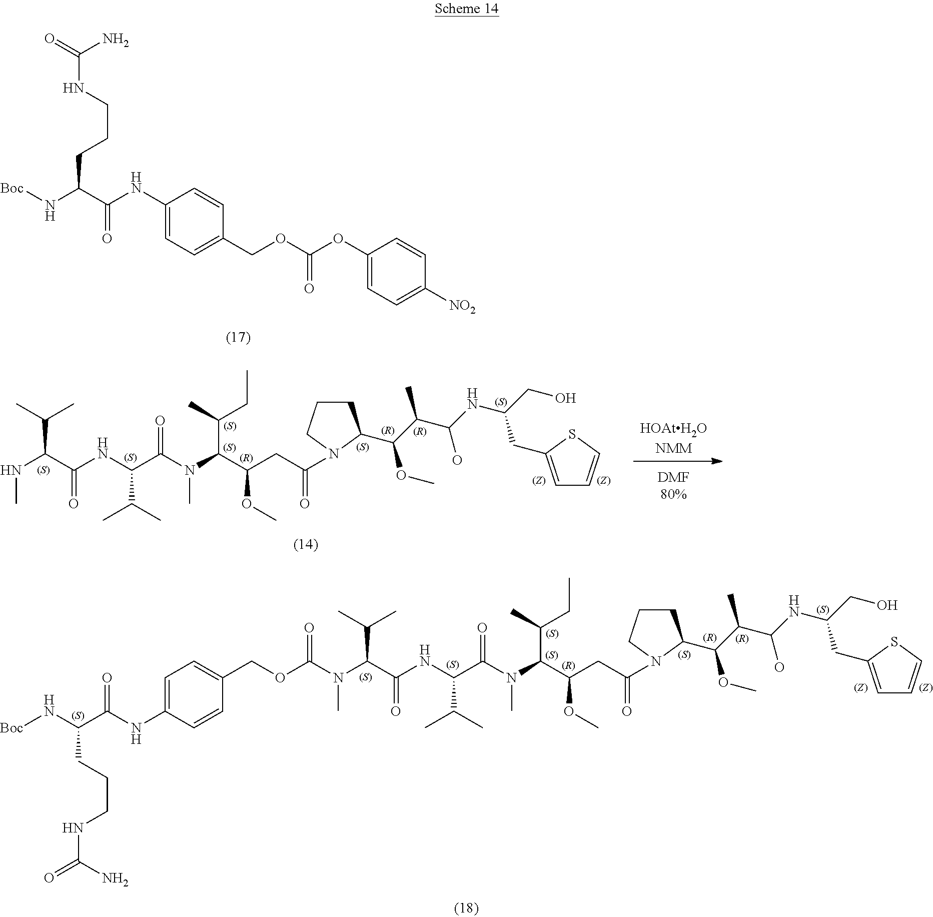

Referring to the reaction outlined in Scheme 14, to a stirred 23° C. solution of Boc-Cit-Paba-PNP (molecule 17; 1.45 g, 2.65 mmol) in DMF (21 mL) was added MMATH (molecule 14; 1.2 g, 1.66 mmol) and HOAt (83.7 mg, 0.55 mmol), and then NMM (0.73 mL, 6.63 mmol). After 72 h the reaction was diluted with EtOAc (200 mL) and washed with 1.0 M HCl (2×100 mL), followed by Sat. NaHCO₃ (1×100 mL) and Brine (1×100 mL). The organic layer was dried with MgSO₄, filtered and concentrated under reduced pressure. The yellow foam was purified by Flash column chromatography on silica gel using 0% to 10% MeOH in EtOAc to give Boc-Cit-Paba-MMATH (molecule 18) as a white foam in 80% yield.

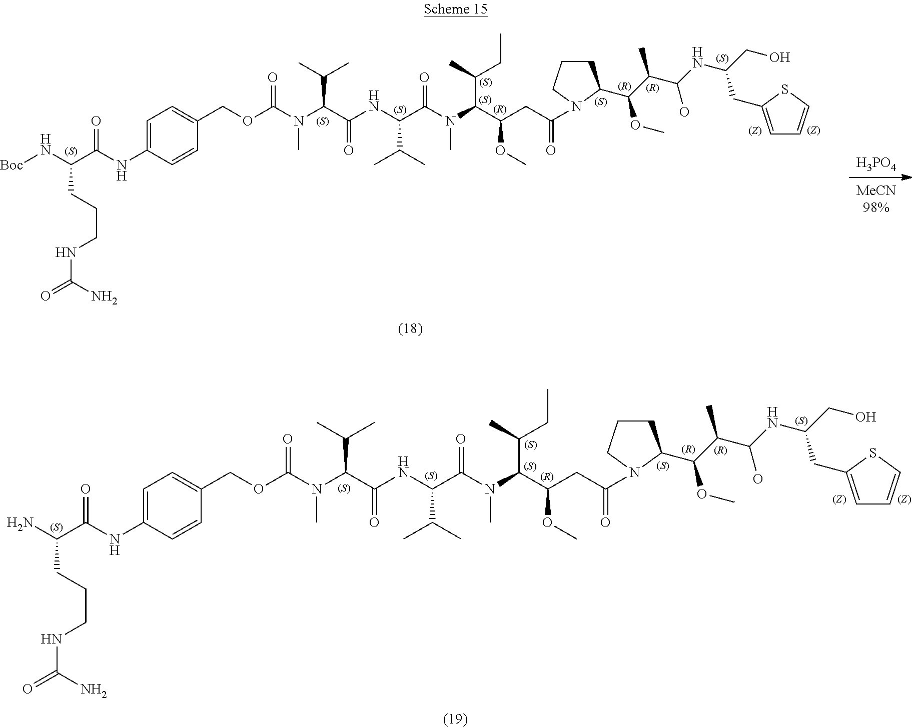

Referring to the reaction outlined in Scheme 15, Boc-Cit-Paba-MMATH (molecule 18, 1.2 g, 1.06 mmol) is dissolved in MeCN (6 mL) using 5 min of sonication. To a stirred 23° C. solution of Boc-Cit-Paba-MMATH (molecule 18; 1.2 g, 1.06 mmol) in MeCN (6 mL) is added H₃PO₄ (6 mL). After 16 h, the solution was diluted with water (15 mL) and adjusted to pH 8 with 10 M aq NaOH. The aqueous layer was extracted with DCM (2×100 mL). The combined organics were dried with MgSO₄, filtered and concentrated under reduced pressure to yield Cit-Paba-MMATH (molecule 19) as yellow foam in 98% yield.

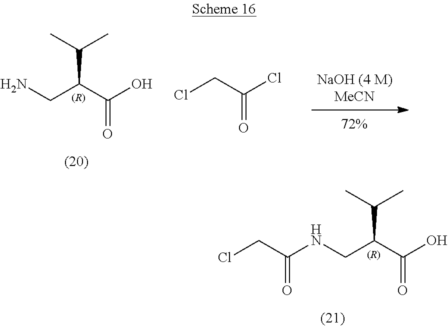

Referring to the reaction outlined in Scheme 16, to a stirred 0° C. suspension of β-homoVal (molecule 20; 1000 mg, 7.62 mmol) in MeCN (40 mL) was added 4 M NaOH (3.81 mL, 15.25 mmol) followed by slow addition (1 mL/min) of dilute ClAcCl (0.60 mL, 7.55 mmol) in MeCN (10 mL). After 20 min, the reaction was diluted with 1 M HCl (100 mL) and EtOAc (100 mL). The aqueous layer was removed and the organic layer washed with 1 M HCl (3×100 mL) followed by brine (1×100 ml). The organic layer was dried with MgSO₄, filtered and concentrated under reduced pressure. The crude reaction was purified by RP-HPLC with a Phenomex Gemini-NX column using 5% to 98% MeCN in 0.05% aqueous TFA as the eluent. Molecule 21 was obtained as a colorless oil (1.14 g).

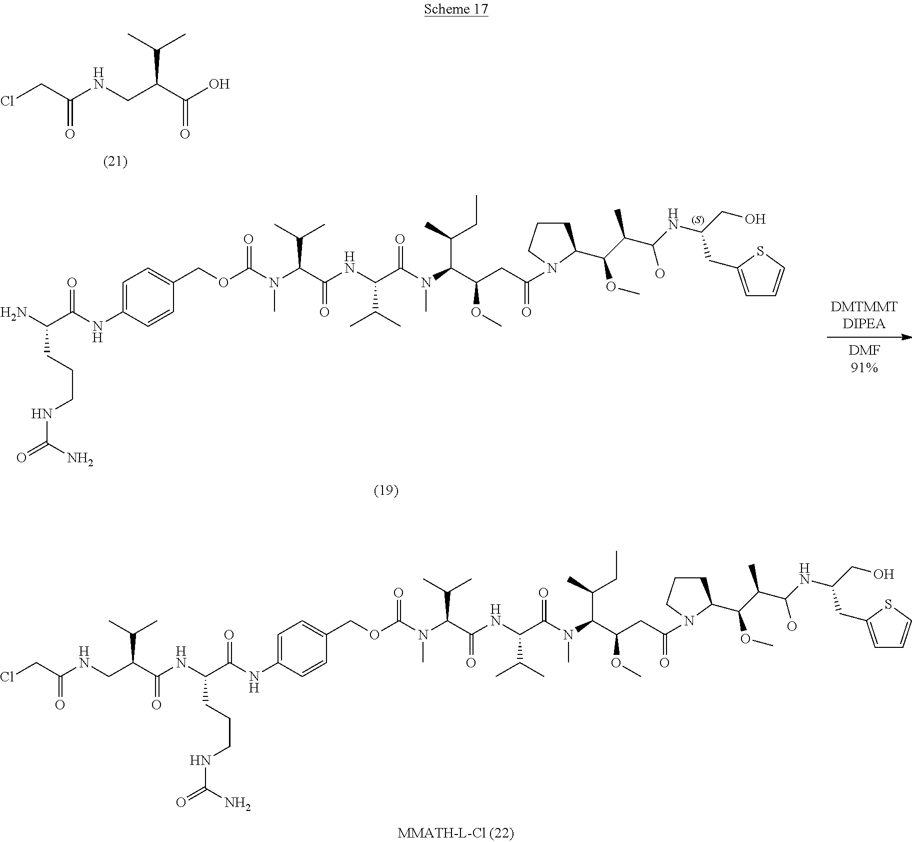

Referring to the reaction outlined in Scheme 17, to a stirred 0° C. solution of DMTMMT (55 mg, 0.14 mmol) in DMF (0.5 mL) was added DIPEA (100 μL, 0.57 mmol) followed by H2N-Cit-Paba-MMATH (molecule 19; 105 mg, 0.1 mmol). After stirring the reaction for 5 min, ClAc-β-homoVal (molecule 21; 30 mg, 0.14 mmol) was added. After 1 h, the crude solution was purified preparatory RP-HPLC with a Phenomenex Gemini 10μ, C18 110 Å column using 5% to 98% MeCN in 0.05% aqueous TFA as the eluent. MMATH-L-Cl (molecule 22) was obtained as a white powder (114 mg, 91%).

In other embodiments of the present disclosure, a MMATH linker-toxin combination includes a bromo- and iodo-derivative of molecule 22, where the chloro group is replaced with a bromo group (molecule 23) or an iodo group (molecule 24), where “Payload” represents a toxin. In some embodiments of the present disclosure, the toxin is MMATH (molecule 22) connected via the N-terminal nitrogen.

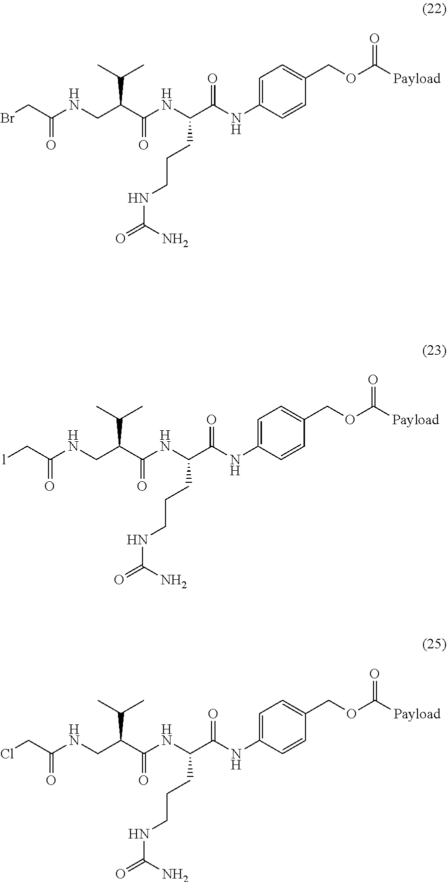

In some embodiments of the present disclosure, the Payload of molecules 23, 24, or 25 can be represented by an agent, such as a toxin.

In some embodiments of the present disclosure, a compound is represented by molecule 26, wherein Payload represents an agent, such as a toxin, and R represents a target-binding moiety, such as an antibody or antigen-binding thereof, or any other molecule via a free thiol group.

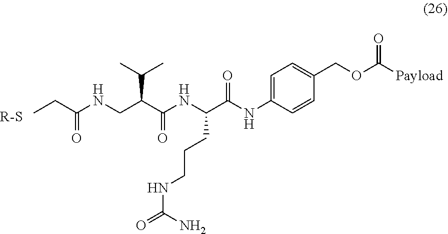

### Example 3: In Vitro Cytotoxicity of Auristatin Species

In this exemplary study, an in vitro cytotoxicity was used to evaluate the relatively toxicity of MMATIH (a thiophenylmethyl hydroxymethyl derivative of an auristatin), as shown herein as formula (I), an auristatin species of the present disclosure.

In this assay, the test cells were plated and grown to an appropriate cell density (e.g., 1500 cells/well (50 μL per well) for SW780 cells). The cells were treated with the drug (MMATH or MMAE (monomethylauristatin E)) at concentrations ranging from 10 μM to 10⁻⁴ nM in triplicate for 5 days. On the day 6 endpoint, the cells were incubated with 20 μL of Presto Blue @ 37 C for 2 hr and the signal was read on a Biotek synergy H4 plate reader. After media background was subtracted, the percent survival was calculated and plotted to determine the EC50, as shown in the exemplary results of Table 1.

TABLE 1
In Vitro Cytotoxicity of Auristatin Species

| | EC50 (nM) | | | | |
|---|---|---|---|---|---|
| Toxin | SW780 | SKOV3 | SW780 | HCC1954 | PC3 |
| MMAE | 0.31 | 0.19 | 0.22 | 0.03 | 0.24 |
| MMATH | 0.38 | 0.20 | 0.26 | 0.03 | 0.24 |

In an exemplary study, the binding of MMAE and MMATH to tubulin was measured, showing a K_{D} of 69.9 nM (MMAE) and 204.4 nM (MMATH). The exemplary results show that the novel MMATH auristatin species has a comparable toxicity to MMAE. These exemplary results also show that this comparable in vitro efficacy was achieved with a molecule with a lower affinity to tubulin, its presumed molecular target for efficacy.

### Example 4: Stability of Linker Species

In this exemplary study, an in vitro study was used to evaluate the stability of a linker (molecule 26) of the present disclosure compared to a valine-citrulline (vc) linker.

In this study, relative kinetic rates of cleavage by a variety of cathepsins for two different cysteine-conjugated drug linkers were measured by LCMS. The enzymes (cathepsins B, D, H, K, L, and S) were activated prior to introduction to substrate. One substrate was the auristatin MMAE linked to cysteine via a valine-citrulline-PAB-carboxy linker (CAS No.: 646502-53-6) and the other was the MMATH auristatin of the present disclosure (molecule 14) linked to a cysteine via a linker of molecule 26.

Two different thiol-linked cysteine-linked auristatins (MMATH-L-Cys and Cys-vc-MMAE) were incubated at 37° C. with pre-activated enzymes over a 48 h time period. Timepoints were aliquoted directly into 2 M, pH 9 Tris buffer to stop enzymatic activity and then immediately frozen to −80° C. AUCs of MS XICs of both free drug and cysteine-linked drug for each were monitored over time. All samples were run on a Thermo LTQ Velos OrbiTrap mass spectrometer using a Dionex LC front end. The amounts of original cysteine-linked drug, free drug, and cleaved “linker” stubs were measured over time.

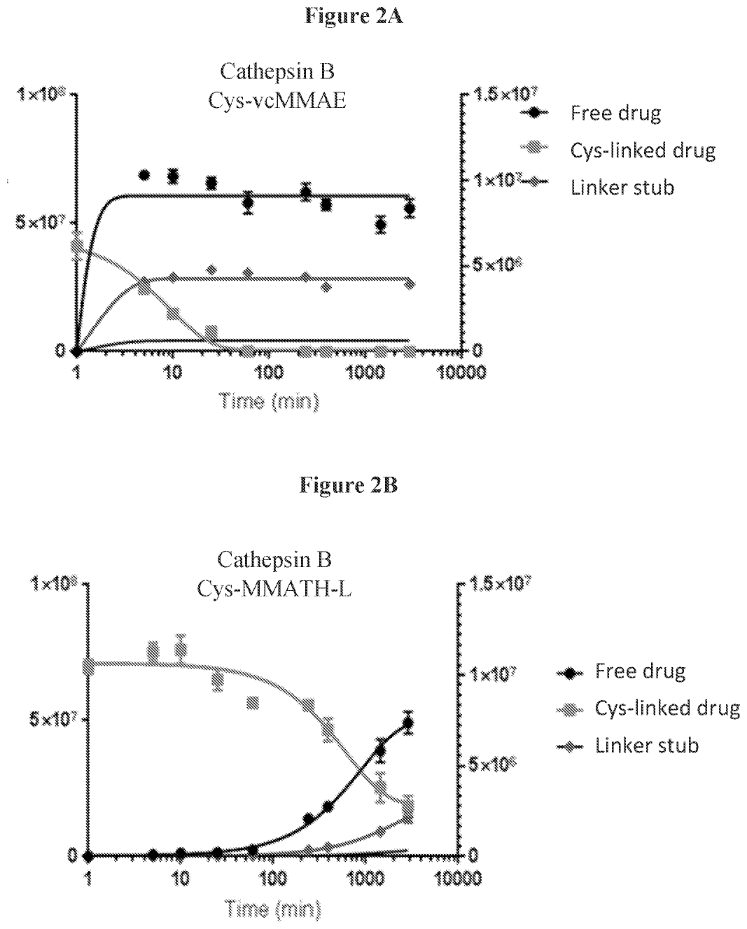

Referring to FIGS. 2A and 2B, the exemplary results show that a cysteine-linked to MMATH with the linker of molecule 26 showed a higher stability than the corresponding cysteine-linked to vcMMAE. In results with cathepsins H, D, L, K, and S, the results showed a similar relatively stability between the two linkers for all cathepsins. Cathepsin D and L cleaved at a rate comparable to cathepsin B, while cathepsin H cleaved relatively slower than cathepsin B. Cathepsins K and S cleaved relatively faster than cathepsin B.

In another exemplary study, the stability of the two cysteine-linked auristatin species were tested in an activated lysosome-derived lysate. In this study, lysosomes were lysed by three consecutive freeze/thaw cycles, followed by 30 min of sonication. The cysteine-linked auristatins were incubated at 37° C. with pre-activated lysosomes over a 24 h time period. In this study, the cysteine-MMATH-L substrate was incubated with a 5× lysosome concentration. Timepoints were taken throughout the incubation and AUCs of MS XICs for both free drug and cys-DL were monitored over time. All samples were run on a Thermo LTQ Velos OrbiTrap mass spectrometer using a Dionex LC front end. The amounts of original cysteine-linked drug, free drug, and cleaved “linker” stubs were measured over time.

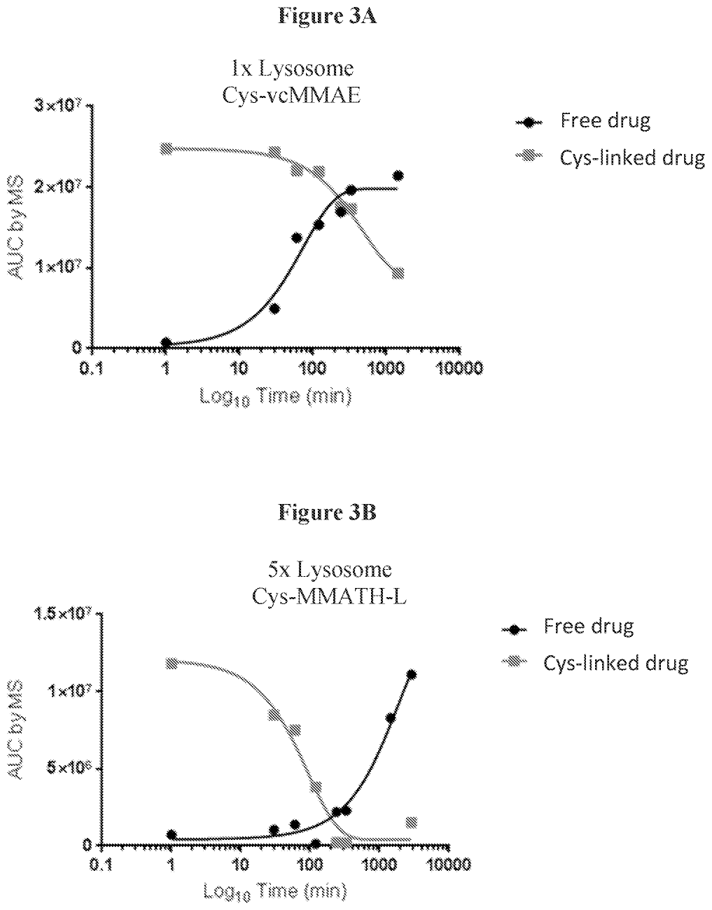

Referring to FIGS. 3A and 3B, the exemplary results show that a cysteine-linked to MMATH with the linker of molecule 26 showed a comparable stability in activated lysosomes than the corresponding cysteine-linked to vcMMAE, even with the former having been treated with a 5× lysosomal concentration, thus indicating about a 5× slower rate of cleavage than vcMMAE linker.

In a further exemplary study, the stability of the substrates were determined in the presence of four different carboxylesterases (human or mouse CES-1 and CES-1C). In this study, the enzymes were activated prior to substrate introduction and then incubated with the substrate at 37° C. over a 48 h time period. Timepoints were aliquoted directly into 2 M, pH 9 Tris buffer to stop enzymatic activity and then immediately frozen to −80° C. AUCs of MS XICs of both free drug and cysteine-linked drug for each were monitored over time. All samples were run on a Thermo LTQ Velos OrbiTrap mass spectrometer using a Dionex LC front end. The amounts of original cysteine-linked drug, free drug, and cleaved “linker” stubs were measured over time.

In this study using CES-1 or CES-1C mouse or human carboxylesterases, no cleavage of either substrate was observed by human or mouse CES-1. However, cysteine-vcMMAE was completely cleaved in 48 hrs by both human and mouse CES-1C. For the MMATH-linker of the present disclosure, cleavage by mouse or human CES-1C begin around 12 hours, and at a rate substantially slower than that observed with vcMMAE.

These exemplary results show that the linker of molecule 26 has a higher stability than the valine-citrulline linker in both activated enzymes and lysosomes. These exemplary results show that the linker-MMATH of molecule 26 has a higher stability than the vcMMAE in both activated enzymes and lysosomes.

### Example 5: Novel Antibody Conjugation Site

In this exemplary study, a leucine residue located in the FG-loop of the human IgG1 heavy chain constant region. For reference, the leucine in question is found in the context of the sequence KVSNKALPAPI (i.e., position 328 Kabat numbering). In the present disclosure, the leucine at this position was site-specifically modified to cysteine, i.e., KVSNKACPAPI.

In this study, the monoclonal antibody trastuzumab, which specifically binds the target HER2, was modified at this position from leucine to cysteine to determine the suitability for drug conjugation and other effects. A comparison between the native trastuzumab and the modified version of the present disclosure is presented below.

| | trastuzumab | trastuzumab (L328C) |
|---|---|---|
| Drag-Antibody Ratio (DAR) | 2 | 1.7 |
| % Unconjugated | 36% | 1% |
| % Aggregated | 5% | 8% |
| In vivo Efficacy of ADC | | 8/8 |
| FcγR1 binding (pM) | 239 | 701 (2.93× reduction) |
| CI q decrease | 1.02× reduction | 3.4× reduction |

These exemplary results showed a significant decrease in the binding to the Fcγ receptors of the L328C variant of the present disclosure as compared to the original antibody, while resulting in a specific DAR of approximately 2, yet resulting in a highly efficient conjugation i.e. less than 1% conjugated antibody.

Other Examples of this Mutations as Described Herein (Anti-PSMA and Anti-SLC34A2 Antibodies)

These exemplary results demonstrate the advantages of using antibodies or activatable antibodies with this site-specific modification, to provide an efficient, controlled site for conjugation with a specific stoichiometry.

### Example 6: Exemplary Method of Conjugation

In this example, an exemplary conjugation method is described to conjugate an auristatin MMATH of the present disclosure to an antibody molecule.

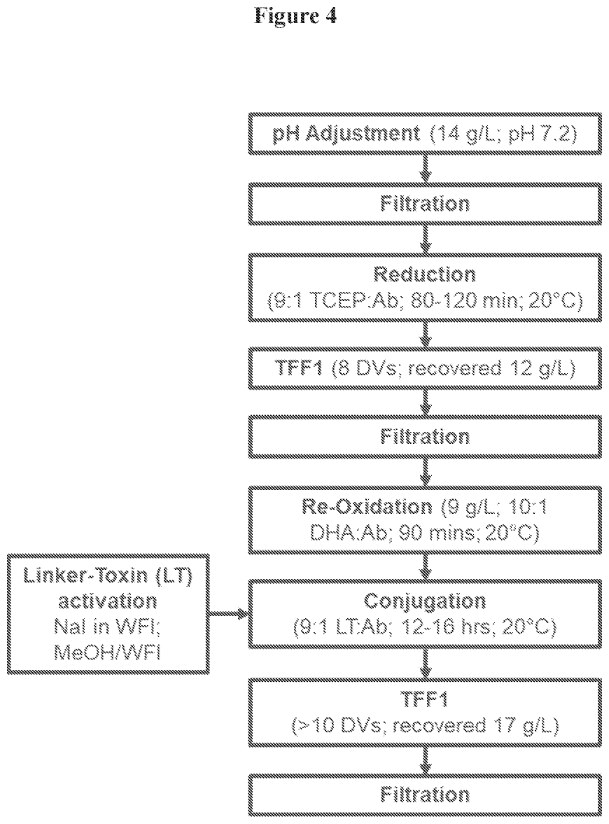

Referring to the exemplary process flow diagram of FIG. 4, in an exemplary method of the present disclosure, an antibody having a cysteine residue at Kabat position 328 is provided at a concentration of 14 g/L at a pH of 7.2. The antibody solution is filtered and then reduced with the reducing agent tris(2-carboxyethyl)phosphine (TCEP) at a 9:1 TCEP:antibody molar ratio for 80-120 minutes at 20° C. The reaction was filtered by tangential flow filtration (TFF) at 8 diavolumes and recovered at 12 g/L. The antibody was re-oxidized with (L)-dehydroascorbic acid (DHA) at a 10:1 DHA:antibody molar ratio for 90 minutes at 20° C.

The MMATH linker-toxin compound having a formula (III), where R2 is a chlorine, was activated with sodium iodide. The activated linker-toxin was added to the re-oxidized antibody at a 9:1 linker-toxin:antibody molar ratio for 12-16 hours at 20° C. to allow conjugation of the linker-toxin to the antibody. The reaction mixture was filtered by TFF at 10 diavolumes and recovered at 17 g/L. Analysis of the conjugated antibody showed site-specific conjugation at the Kabat 328 cysteine positions with a DAR of 2.

These exemplary results showed that the auristatin derivatives of the present disclosure can be conjugated to an antibody in a site-specific manner to provide an antibody-drug conjugate with a DAR of 2.

These exemplary results also showed that by conjugating the linker-toxin to a site-specific cysteine at Kabat position 328, the conjugation can proceed using an iodine-activated coupling of the linker-toxin to the cysteine thiol group. In this manner, the conjugated product is less susceptible to deconjugation reactions than thiol-maleimide conjugates, the latter of which can more readily be reversed by thiol exchange, resulting in an undesirable release of the linker-toxin. The use of antibodies with site-specific cysteines for linker-toxin conjugation, such as those at Kabat position 328, also provide a conjugated antibody product with a DAR of 2. The use of such antibodies with site-specific cysteine residues, such as those at Kabat position 328, also allow linker-toxin conjugation to the antibody without disruption of the native intra- or interchain disulfide bonds of the antibody.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following.

What is claimed is:

1. A compound of formula (I): 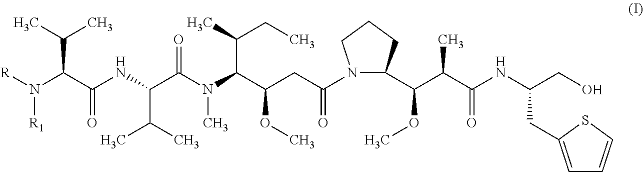
wherein R1 is a hydrogen or a C₁₋₆ alkyl group;
wherein R is selected from the group consisting of: a hydrogen, a C₁₋₆ alkyl, a linker, or a group X1-Y1-* wherein * is the point of attachment to the nitrogen; and
wherein Y1 is an oxycarbonyl group and X1 is a C₁₋₆ alkyl group, a 9-fluorenylmethyl group, a benzyl group, or a tert-butyl group.

2. The compound of claim 1, wherein R1 is a methyl group and R is a hydrogen.

3. The compound of claim 1, wherein X1-Y1 is a 9-fluorenylmethoxycarbonyl (Fmoc) group.

4. A compound of formula (II): 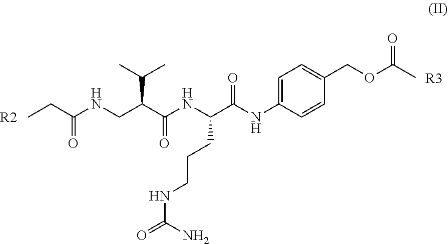
wherein R3 is an agent attached to formula (II) where the point of attachment is a nitrogen, sulfur, oxygen, or carbon atom; and
wherein R2 is a moiety attached to formula (II) wherein the point of attachment is selected from the group consisting of: a chlorine group, an iodine group, a bromine group, and a thiol group.

5. The compound of claim 4, wherein R2 is a target-binding moiety, wherein the point of attachment at R2 is a thiol group.

6. The compound of claim 5, wherein the thiol group is a side chain thiol group of a cysteine residue.

7. A compound of formula (III): 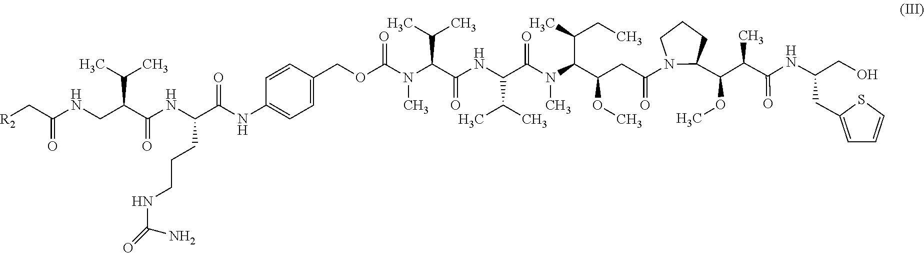
wherein R2 is a moiety attached to formula (III) wherein the point of attachment is selected from the group consisting of: a chlorine group, an iodine group, a bromine group, and a thiol group.

8. The compound of claim 7, wherein R2 is a target-binding moiety, wherein the point of attachment at R2 is a thiol group.

9. The compound of claim 8, wherein the thiol group is a cysteine side chain thiol group.

10. The compound of any one of claims 4 to 9, wherein the target-binding moiety is an isolated antibody or an antigen binding fragment thereof (AB) that specifically binds to the target.

11. The compound of any one of claims 4 to 9, wherein the target-binding moiety is an activatable antibody that, in an activated state, specifically binds to the target, the activatable antibody comprising:
an antibody or an antigen binding fragment thereof (AB) that specifically binds to the target;
a masking moiety (MM) coupled to the AB, wherein the MM inhibits the binding of the AB to the target when the activatable antibody is in an uncleaved state; and
a cleavable moiety (CM) coupled to the AB, wherein the CM is a polypeptide that functions as a substrate for a protease.

12. The compound of claim 11, wherein the MM has a dissociation constant for binding to the AB that is greater than the dissociation constant of the AB to its target.

13. The compound of claim 11 or claim 12, wherein the MM does not interfere or compete with the AB for binding to its target when the activatable antibody is in a cleaved state.

14. The compound of any one of claims 11 to 13, wherein the MM is a polypeptide of no more than 40 amino acids in length.

15. The compound of any one of claims 11 to 14, wherein the MM polypeptide sequence is different from that of the target sequence.

16. The compound of any one of claims 11 to 15, wherein the MM polypeptide sequence is no more than 50% identical to any natural binding partner of the AB.

17. The compound of any one of claims 10 to 16, wherein the target is selected from the group consisting of CD44, CD147, CD166, ITGa3, ITGb1, PSMA, and SLC34A2.

18. The compound of any one of claims 4 to 6, wherein the agent is selected from the group consisting of auristatin E, monomethyl auristatin F (MMAF), monomethyl auristatin E (MMAE), monomethyl auristatin D (MMAD), maytansinoid DM4, maytansinoid DM1, a calicheamicin, a duocarmycin, a pyrrolobenzodiazepine, and a pyrrolobenzodiazepine dimer.

19. The compound of any one of claims 1 to 3, wherein R is a linker.

20. The compound of claim 19, wherein the linker is a cleavable linker.

21. The compound of claim 19 or claim 20, wherein the linker is linked to a target-binding moiety.

22. The compound of claim 21, wherein the target-binding moiety is an isolated antibody or an antigen binding fragment thereof (AB) that specifically binds to the target.

23. The compound of claim 21, wherein the target-binding moiety is an activatable antibody that, in an activated state, specifically binds to the target, the activatable antibody comprising:
an antibody or an antigen binding fragment thereof (AB) that specifically binds to the target;
a masking moiety (MM) coupled to the AB, wherein the MM inhibits the binding of the AB to the target when the activatable antibody is in an uncleaved state; and
a cleavable moiety (CM) coupled to the AB, wherein the CM is a polypeptide that functions as a substrate for a protease.

24. The compound of any one of claims 21 to 23, wherein the target is selected from the group consisting of CD44, CD147, CD166, ITGa3, ITGb1, PSMA, and SLC34A2.

25. The compound of any one of claims 10 to 23, wherein the antibody or activatable antibody comprises a cysteine residue at Kabat position 328.

26. An IgG1 antibody, wherein position Kabat position 328 is a cysteine.

27. An activatable antibody comprising:
an antibody or an antigen binding fragment thereof (AB) that specifically binds to the target;
a masking moiety (MM) coupled to the AB, wherein the MM inhibits the binding of the AB to the target when the activatable antibody is in an uncleaved state; and
a cleavable moiety (CM) coupled to the AB, wherein the CM is a polypeptide that functions as a substrate for a protease,
wherein position Kabat position 328 of the AB is a cysteine.

28. The antibody or claim 26 or the activatable antibody of claim 27, wherein the antibody or the AB specifically binds to a target selected from the group consisting of CD44, CD147, CD166, ITGa3, ITGb1, PSMA, and SLC34A2.

29. A pharmaceutical composition comprising:
the compound, antibody, or activatable antibody of any one of claims 1 to 28; and a suitable carrier.

30. A method of conjugating a compound to a polypeptide, the method comprising:
conjugating a compound of formula (I) to a polypeptide: 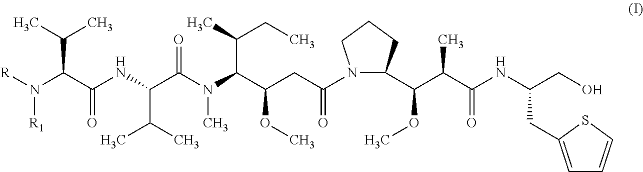
wherein R1 is a hydrogen or a C₁₋₆ alkyl group;
wherein R is selected from the group consisting of: a hydrogen, a C₁₋₆ alkyl, a linker, or a group X1-Y1-* wherein * is the point of attachment to the nitrogen; and
wherein Y1 is an oxycarbonyl group and X1 is a C₁₋₆ alkyl group, a 9-fluorenylmethyl group, a benzyl group, or a tert-butyl group;
wherein at least one equivalent of the compound of formula (I) or a derivative thereof is conjugated to the polypeptide.

31. The method of claim 30, wherein R1 is a methyl group and R is a hydrogen.

32. The method of claim 30, wherein X1-Y1 is a 9-fluorenylmethoxycarbonyl (Fmoc) group.

33. The method of claim 30, wherein R is a linker.

34. The method of claim 33, wherein the linker is a cleavable linker.

35. A method of conjugating a compound to a polypeptide, the method comprising:
conjugating a compound formula (III) to a polypeptide:
(III), 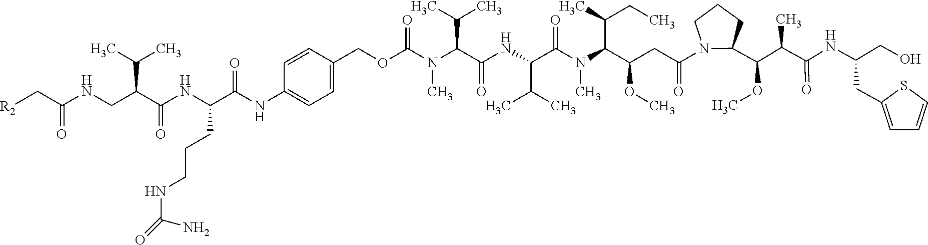
wherein R2 is a moiety attached to formula (III) wherein the point of attachment is selected from the group consisting of: a chlorine group, an iodine group, a bromine group, and a thiol group.

36. The method of claim 35, wherein the R2 is a halogen group.

37. The method of claim 36, wherein the R2 is an iodine group,

38. The method of claim 36, wherein the R2 is a bromine group.

39. The method of claim 36, wherein the R2 is a chlorine group.

40. The method of any one of claims 30 to 39, wherein at least one compound of formula (I) or (III) is conjugated to the polypeptide via a thiol group on the polypeptide.

41. The method of claim 40, wherein the thiol group is a side chain thiol group of a cysteine residue of the polypeptide.

42. The method of any one of claims 30 to 41, wherein the polypeptide comprises a target-binding moiety.

43. The method of any one of claims 30 to 42, wherein the polypeptide comprises an antibody or an antigen binding fragment thereof (AB) that specifically binds to a target.

44. The method of claim 43, wherein the cysteine residue is at Kabat position 328 of the AB.

45. The method of any one of claims 30 to 44, wherein the method comprises the steps of:
(i) reducing the polypeptide with a reducing agent, wherein at least one disulfide group is reduced to a free thiol group;
(ii) re-oxidizing the polypeptide with an oxidizing agent without oxidizing the free thiol group; and
(iii) conjugating the compound of formula (I) or (III) to the free thiol group.

46. The method of claim 45, wherein the reducing agent is TCEP.

47. A conjugated polypeptide having the formula:
[T]-[L]-[C];
wherein [T] is a target-binding moiety and [L] is a linker moiety; and
wherein[C] is a compound comprising a compound of formula (I): 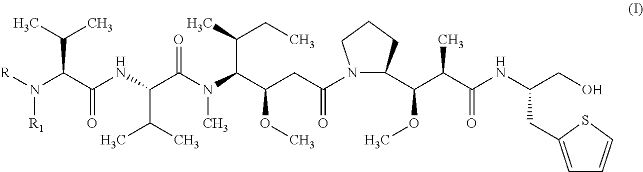
wherein R1 is a hydrogen or a C₁₋₆ alkyl group; and
wherein R is the point of attachment to [L].

48. The conjugated polypeptide of claim 47, wherein R1 is a methyl group.

49. A conjugated polypeptide having the formula:
[T]-[LC];
wherein [T] is a target-binding moiety and [LC] is a linker-toxin; and
wherein [LC] is a compound comprising a compound of formula (III): 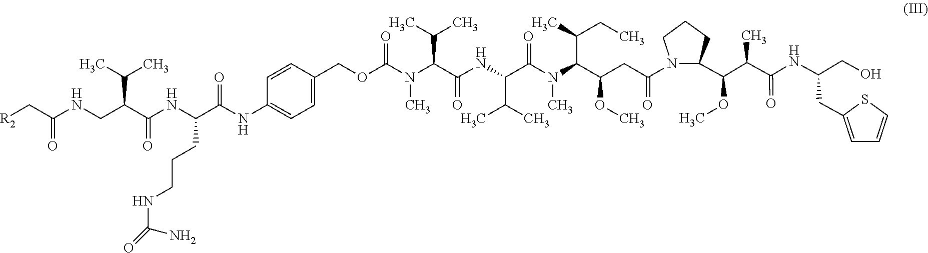
wherein R2 is the point of attachment to [T].

50. The conjugated polypeptide of claim 47 or claim 48, wherein the linker [L] is a cleavable linker.

51. The conjugated polypeptide of any one of claims 47 to 50, wherein the linker [L] or the linker-toxin [LC] is coupled to the target-binding moiety [T] via a thiol group on the target-binding moiety.

52. The conjugated polypeptide of claim 51, wherein the thiol group is a side chain thiol group of a cysteine residue on the target-binding moiety.

53. The conjugated polypeptide of any one of claims 47 to 52, wherein the target-binding moiety [T] comprises an antibody or an antigen binding fragment thereof (AB) that specifically binds to a target.

54. The conjugated polypeptide of claim 53, wherein the cysteine residue is a cysteine residue at Kabat position 328 of the AB.

55. A method of treating a subject with a disease or disorder comprising:
administering to a subject in need thereof an effective amount of a composition comprising the compound of any one of claims 1 to 25, the pharmaceutical composition of claim 29, or the conjugated polypeptide of any one of claims 47 to 54.

56. Use of a compound of any one of claims 1 to 25, a pharmaceutical composition of claim 29, or a conjugated polypeptide of any one of claims 47 to 54 for treating a disease or disorder.

57. A compound of any one of claims 1 to 25, a pharmaceutical composition of claim 29, or a conjugated polypeptide of any one of claims 47 to 54 for use in the preparation of a medicament for treating a disease or a disorder.

58. The method of any one of claims 55 to 57, wherein the disease or disorder is a cancer.
